SEQUENCE LISTING (11 sequences)

SEQ ID NO: 1 - DNA, 15 bp, synthetic construct
  misc_feature 1..15: note = Description of sequence: T_sub
  misc_feature 1: note = Modified_base - Description: 5-Carboxytetramethylrhodamine (fluorophore) at 5'
  misc_feature 15: note = Modified_base - Description: Black Hole Quencher-2
  misc_feature 9: note = n is ribose spacer (RNA) having 2 nucleotides of: gu
  source 1..15: mol_type = other DNA
gtttcctcnccctgg

SEQ ID NO: 2 - DNA, 30 bp, synthetic construct
  misc_feature 1..30: note = Description of sequence: Dz1e
  misc_feature 1..30: note = Additional description: DNAzyme
  source 1..30: mol_type = other DNA
ccagggaggctagctacaacgagaggaaac

SEQ ID NO: 3 - DNA, 56 bp, synthetic construct
  misc_feature 1..56: note = Description of sequence: IDz1
  misc_feature 33: note = n is ribose spacer (RNA) nucleotides having 2 nucleotides of: gu
  source 1..56: mol_type = other DNA
ccagggaggctagctacaacgagaggaaacacncatcagggaaaagcctccctaaa

SEQ ID NO: 4 - DNA, 31 bp, synthetic construct
  misc_feature 1..31: note = Description of sequence: Dz2
  misc_feature 1..31: note = Additional description: DNAzyme
  source 1..31: mol_type = other DNA
cctgatgaggctagctacaacgagtgtttcc

SEQ ID NO: 5 - DNA, 26 bp, synthetic construct
  misc_feature 1..26: note = Description of sequence: Dza2
  misc_feature 1..26: note = Additional description: DNAzyme
  source 1..26: mol_type = other DNA
aaccacacaacacaacgagtgtttcc

SEQ ID NO: 6 - DNA, 27 bp, synthetic construct
  misc_feature 1..27: note = Description of sequence: Dzb2
  misc_feature 1..27: note = Additional description: DNAzyme
  source 1..27: mol_type = other DNA
cctgatgaggctagctctactacctca

SEQ ID NO: 7 - DNA, 22 bp, synthetic construct
  misc_feature 1..22: note = Description of sequence: let-7b
  misc_feature 1..22: note = Additional description: DNAzyme
  source 1..22: mol_type = other DNA
tgaggtagtaggttgtgtggtt

SEQ ID NO: 8 - DNA, 21 bp, synthetic construct
  misc_feature 1..21: note = Description of sequence: T1
  misc_feature 1..21: note = Additional description: DNAzyme
  source 1..21: mol_type = other DNA
cacacgtgttctgttcgtcta

SEQ ID NO: 9 - DNA, 76 bp, synthetic construct
  misc_feature 1..76: note = Description of sequence: T2
  misc_feature 28: note = n is Int Spacer 9 (iSp9)
  misc_feature 50: note = n is Int Spacer 9 (iSp9)
  source 1..76: mol_type = other DNA
cctgatgaggctagctctactacctcantagacgaacgaaacacgtgtgnaaccacacaacacaacgagtgtttcc

SEQ ID NO: 10 - RNA, 26 nt, synthetic construct
  misc_feature 1..26: note = Description of sequence:
  modified_base 1: mod_base = OTHER; note = Modified_base - Description: Modification: 6-FAM (Fluorescein) at 5'
  source 1..26: mol_type = other RNA
cagtcgactcgccgattagacgaaca

SEQ ID NO: 11 - DNA, 26 bp, synthetic construct
  misc_feature 1..26: note = Description of sequence: TAMRA-DNA
  modified_base 1: mod_base = OTHER; note = Modified_base - Description: 5-Carboxytetramethylrhodamine (fluorophore) at 5'
  source 1..26: mol_type = other DNA
cagtcgactcgccgattagacgaaca

(19) United States
(12) Patent Application Publication
(10) Pub. No.: US 2025/0051833 A1
(43) Pub. Date: Feb. 13, 2025

(54) SUPRAMOLECULAR ENCAPSULATED DNA/RNA PARTICLES

(71) Applicant: YEDA RESEARCH AND DEVELOPMENT CO. LTD., Rehovot (IL)

(72) Inventors: Sergey N. SEMENOV, Rehovot (IL); Ekaterina V. SKORB, Rehovot (IL)

(73) Assignee: YEDA RESEARCH AND DEVELOPMENT CO. LTD., Rehovot (IL)

(21) Appl. No.: 18/721,921

(22) PCT Filed: Dec. 26, 2022
(86) PCT No.: PCT/IL2022/051389

§ 371 Date: Jun. 20, 2024

Related U.S. Application Data
(60) Provisional application No. 63/293,776, filed on Dec. 26, 2021.

(51) Int. Cl. C12Q 1/6834; C12Q 1/6876

(52) U.S. Cl. CPC C12Q 1/6834; C12Q 1/6876

(57) ABSTRACT

Provided herein a supramolecular assembly comprising a solid porous particle comprising a single-stranded polynucleotide encapsulated by non-covalent organic structure. Specifically, the non-covalent organic structure is melamine-cyanurate, and the single-stranded polynucleotide is ssDNA or ssRNA.

### SEQUENCE LISTING

The instant application contains a Sequence Listing which has been submitted electronically in XML file format and is hereby incorporated by reference in its entirety. Said XML copy, created on Dec. 26, 2022, is entitled “P-621441-PC_ST26.xml”, and is 21,142 bytes in size.

### FIELD OF THE INVENTION

Provided herein a supramolecular assembly comprising a solid porous particle comprising a single-stranded polynucleotide encapsulated by non-covalent organic structure. Specifically, the non-covalent organic structure is melamine-cyanurate, and the single-stranded polynucleotide is ssDNA or ssRNA.

### BACKGROUND OF THE INVENTION

DNA polynucleotides (ssDNA) are used in many applications, such as primers and probes in diagnostics, biosensing, and bioanalysis. The strict requirements for ssDNA storage conditions limit its applicability in many studies. Generally, ssDNA storage goes through a few preliminary costly procedures such as freeze-drying. In case of a long-term storage, the most important requirement is for a constant low temperature. The buffer solution, essential for most implementations, also affects ssDNA if it is undried. Therefore, there remains a need for a more simplified procedure for ssDNA storage.

Approaches for ssDNA storage involve incorporating ssDNA into protecting shells such as lipids or hybrid particles. One of the most effective techniques is layer-by-layer ssDNA loading. It provides the DNA loading through cyclic bilayer formation and, finally, protection shell formation. Despite the high percent of ssDNA loaded, the procedure is time consuming. Besides, several difficult procedures such as moderate heat-treating combined with high humidity can trigger the shell destruction and DNA release. Several solid hosts can load DNA without bicyclic layer formation, such as cationic nanoparticles. These solid hosts provide protection without any extra protective outer layer. Thus, these solid hosts are chosen specifically to an encapsulated substance and hardly applicable for a wide range of reagents.

Another approach is post-formation ssDNA-loading, typically performed by metal-organic frameworks. The main requirement is a positive charge of the metal-organic framework to provide DNA-loading. These frameworks effectively capture ssDNA from solutions and store it in pores, but the DNA release requires immersing the material in the solution of complementary DNA strands.

Stimuli-responsive release of an encapsulated compound is one of the most important requirements to the particles with reversible chemical bonding such as hydrogen bonding. Melamine (M) cyanurate (CA) supramolecular assemblies (M-CA) are pH-sensitive, which is a significant advantage for release. Melamine cyanurate (M-CA) is one of the most stable organic self-assemblies. Its structure consists of infinite hexagonally packed layers composed of altering melamine and cyanuric acid molecules (FIG. 1A).

Provided herein is a melamine cyanurate supramolecular core-shell structure that allows the encapsulation of ssDNA or ssRNA by self-assembly, and its use in the detection of polynucleotides.

### SUMMARY OF THE INVENTION

In some embodiments provided herein a supramolecular assembly comprising a solid porous particle comprising a plurality of single-stranded polynucleotides encapsulated by non-covalent organic structure. In other embodiments the porous particle is crystalline, in other embodiments the non-covalent organic structure comprises melamine-cyanurate or melamine-barbiturate or derivative thereof.

In some embodiments, provided herein a method of detecting the presence of a nucleotide sequence in a sample, the method comprising:
a. obtaining a sample;
b. contacting the sample with a supramolecular assembly comprising a solid porous particle comprising a plurality of single-stranded polynucleotides encapsulated by non-covalent organic structure;
c. adjusting the pH of the solution for dissolution of particles; and
d. detecting the presence of a nucleotide sequence.

In some embodiments, provided herein a method of detecting the presence of a nucleotide sequence in a sample, the method comprising:
a. obtaining a sample;
b. contacting the sample with a supramolecular assembly of this invention;
c. adjusting the pH of the solution for dissolution of particles; and
d. detecting the presence of a nucleotide sequence.

In other embodiments, the nucleotide sequence used in the methods of this invention is selected from one or more of DNA, RNA, cDNA, mRNA, CRNA, tRNA, ribosomal RNA, dsDNA, ssDNA, miRNA, siRNA, and mitochondrial DNA.

In some embodiments, provided herein a method of gene suppression in a cell, the method comprising: contacting the cell with a supramolecular assembly of this invention.

In some embodiments, provided herein a method of expressing a gene in a cell, the method comprising: contacting the cell with a supramolecular assembly of this invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A—Molecular structure of melamine cyanurate (M-CA) assembly.

FIGS. 2A-2E present the melamine cyanurate (M-CA) system provided herein. FIG. 2A—presents schematic agar gels containing melamine (M), ssDNA, and cyanuric acid (CA) present in the system (from the left to the right). FIG. 2B—1 wt % agar gels containing 13 mM melamine (M), 100 nM ssDNA, and 13 mM cyanuric acid (CA) in the presence of 50 mM Mg²⁺. The upper image shows melamine cyanurate (M-CA) particles formed within the ssDNA containing gel during the reaction-diffusion process at specially design system. The bottom image shows M-CA particle with incorporated ssDNA in RHOD channel. FIG. 2C—M-CA particles' assembly during reaction-diffusion process and its disassembly after pH change. FIG. 2D—Molecular structures of the components. FIG. 2E—Fluorescence spectrum of the TAMRA fluorophore. FIG. 2F—Representative UV-Vis spectra of TAMRA-DNA showing distinctive absorption peaks for the DNA (1) and TAMRA dye (2).

FIG. 3—M-CA particles in the absence of ssDNA. Scale bar 100 μm.

FIGS. 4A-4D—Particle formation during nucleation and post-nucleation. FIG. 4A—Time-dependent images of the M-CA particles' assembly in bright filed (bottom) and RHOD channel (top) at different reaction times. FIG. 4B—Fluorescence intensity profile during formation of the M-CA particle, the profile is built through the center of the particle. FIG. 4C—Snapshots of molecular dynamics computational cell for Mg²⁺-containing system along the process of ssDNA-M-CA cluster formation (green spheres represent Mg²⁺ ions, water molecules are not shown). FIG. 4D—Scanning electron microscopy images of the particles.

FIGS. 5A-5I—Environmental conditions effect recognition. FIG. 5A—Fluorescence of the particle without Mg²⁺ in RHOD channel. FIG. 5B—Fluorescence of the particle in presence of 50 mM Mg²⁺ in RHOD channel. FIG. 5C—Averaged fluorescence intensity profiles of the particles with and without magnesium in RHOD channel. FIG. 5D-5E—Representative shortcuts from MD simulation of M. CA and DNA assembly in the absence of Mg²⁺ ions (FIG. 5D), and in the presence of Mg²⁺ ions (FIG. 5E) taken at t=0.1 μs. FIG. 5F—Growth curves for molecular clusters formed in the presence (solid black) and in the absence (dashed black) of Mg²⁺, where both lines are overlaying, the gray curve shows a total charge of the cluster in Mg²⁺-containing system. FIG. 5G—Fluorescent and optical microscopy data for VGG16 neural network. FIG. 5H—Confusion matrix for VGG16 neutral network. FIG. 5I—Plots of the ratio of training accuracy and validation accuracy (left), training loss and validation loss (right) for VGG16.

FIGS. 6A-6D—(FIG. 6A) Average amount of the particles in the same section of ssDNA gel (FIG. 6B, rectangle) depending on Mg²⁺ concentrations. Red bars indicate standard deviation, blue bars indicate standard error. (FIG. 6C) Particles size distribution in the mentioned section of ssDNA gel depending on Mg²⁺ concentrations. For each concentration five samples were analyzed. FIG. 6D—Comparison of samples with M-CA particles formed in the absence of 50 mM Mg²⁺ (upper images) and 500 mM of Mg²⁺ (bottom images) after first three hours. Particles form slower in the absence of 500 mM of Mg²⁺. Scale bar 100 μm.

FIGS. 7A-7B—Herman's orientation parameter: FIG. 7A—the average value over the whole molecular cluster during the simulation. FIG. 7B—distribution of orientation parameter along the distance from ssDNA molecule for Mg²⁺-containing system at t=0.6, 0.8 and 0.9 μs. Analysis of the Hermans' orientation parameter demonstrates that for both systems first layers of accommodated M-CA are disordered, but after the cluster reaches the size of 300-400 molecules they become more organized with a pattern similar to a rosette-like one (typical for pure M-CA crystals).

FIG. 8 Dimer existence autocorrelation function. DACF analysis of stability for ssDNA-Mg²⁺. M-Mg²⁺ and CA-Mg²⁺ complexes in solution. Mg²⁺ do not form any long-living complexes with individual M or CA molecules, while its interaction with ssDNA is much more stable.

FIGS. 9A-9D—Transformation of M-CA particles and DNA-cascade running. FIG. 9A—Time-dependent microscopy images of the particle disassembly in RHOD channel (top) and bright field (bottom). FIG. 9B—Changes in the intensity of fluorescence in the center and at edge of the particle and in the background in RHOD channel. Square dots indicate borders of the particles. Panel presents changes in the fluorescence intensity profile during the particle disassembly in RHOD channel. FIG. 9C—Scheme of biochemical reaction between T-sub and DNAzyme (Dz1) with quencher cleavage. FIG. 9D—Time-dependent reaction-diffusion system with 100 nMT-sub containing gel, and reaction-diffusion system with 100 nM T-sub containing gel and 100 nM Dz1 containing gel, both systems in presence of 50 mM Mg²⁺: BF, luminescence in RHOD channel after 1, 4 and 7 days. Scale bar 50 μm.

FIG. 10—melamine cyanurate (M-CA) particles formed with the ssRNA. images of the M-CA particles' assembly in bright filed (bottom) and RHOD channel (top).

FIGS. 11A-11B—Schemes of biochemical reaction between DNAzymes, according to some embodiments. FIG. 11A—biochemical reaction between IDz1 (SEQ ID NO: 3) and Dz2 (SEQ ID NO: 4). FIG. 11B—biochemical reaction between IDz1 (SEQ ID NO: 3) and Dza2 (SEQ ID NO: 5), Dzb2 (SEQ ID NO: 6), let-7b (SEQ ID NO: 7), T1 (SEQ ID NO: 8), and T2 (SEQ ID NO: 9).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Supramolecular Assembly

In some embodiments, provided herein is a supramolecular assembly comprising a solid porous particle comprising a plurality of single-stranded polynucleotides encapsulated by non-covalent organic structure. In some embodiments, provided herein is a supramolecular crystalline assembly comprising a solid porous particle comprising a plurality of single-stranded polynucleotides encapsulated by non-covalent organic structure. In some embodiments, provided herein is a supramolecular assembly comprising a crystalline solid porous particle comprising a plurality of single-stranded polynucleotides encapsulated by non-covalent organic structure. In some embodiments, provided herein is a supramolecular assembly comprising a solid porous particle comprising a core comprising a plurality of single-stranded polynucleotides encapsulated by non-covalent organic structure.

A skilled artisan would appreciate that the terms “nucleic acid sequence”, “nucleotide sequence”, “nucleic acid molecule” or “polynucleotide” may be used interchangeably having all the same qualities and meanings.

In some embodiments, provided herein is a supramolecular assembly comprising a solid porous particle comprising a plurality of single-stranded polynucleotides encapsulated by non-covalent organic structure, wherein the non-covalent organic structure interacts with the single-stranded polynucleotides by intermolecular interactions. In some embodiments, provided herein is a supramolecular assembly comprising a solid porous particle comprising a plurality of single-stranded polynucleotides encapsulated by non-covalent organic structure, wherein the non-covalent organic structure interacts with the single-stranded polynucleotides by hydrogen bonds.

In some embodiments, provided herein is a supramolecular assembly comprising a solid porous particle comprising a plurality of single-stranded polynucleotides encapsulated by non-covalent organic structure, wherein the non-covalent organic structure comprises:
melamine and cyanurate (“melamine-cyanurate”) or derivatives thereof;
melamine and barbiturate (“melamine-barbiturate”) or derivatives thereof;
aromatic amino acids: such as phenylalanine and/or tyrosine;
Fmoc amino acids and Fmoc peptides: such as Fmoc-phenylalanine, Fmoc-tyrosine; Fmoc-asparatic acid, Fmoc-alanine, Fmoc-glutamic acid, Fmoc-glutamine, Fmoc-asparagine, Fmoc-di-phenylalanine or Fmoc-Arg-Gly-AspO or combinations thereof;
diketopiperazines; such as diphenyldiketopiperazine or derivatives thereof; 1,3,5-benzenetricarboxylic acid or derivatives thereof; or
perylene bisimide or derivatives thereof.

In other embodiments the non-covalent organic structure is solid. In other embodiments the non-covalent organic structure is crystalline.

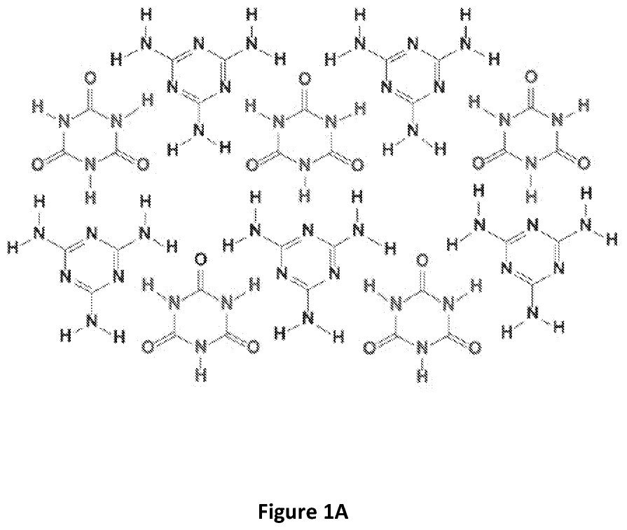

In some embodiments, the non-covalent organic structure comprises melamine-cyanurate (as depicted in FIG. 1A) or derivative thereof. In other embodiments, the molar ratio between the melamine and the cyanurate is 1:1.

In some embodiments, the non-covalent organic structure comprises melamine-barbiturate or derivative thereof. In other embodiments, the molar ratio between the melamine and the barbiturate is 1:1.

In other embodiments, a derivative of melamine refers to a substituted amine (i.e. the melamine includes at least one NHR or N(R)₂ group, wherein R is an alkyl (e.g. methyl, ethyl, isopropyl, tertbutyl), alkenyl, alkynyl, aryl (e.g., phenyl, 4-methylphenyl, naphtyl), cycloalkyl (e.g., cyclohehyl), heterocyclyl (e.g., pyridyl, imidazolyl)). In other embodiments, a derivative of cyanurate or barbiturate refers to a substituted amine (i.e. the barbiturate or cyanurate includes at least one NR′, wherein R′ is an alkyl (e.g. methyl, ethyl, isopropyl, tertbutyl), alkenyl, alkynyl, aryl (e.g., phenyl, 4-methylphenyl, naphtyl), cycloalkyl (e.g., cyclohexyl), heterocyclyl (e.g., pyridyl, imidazolyl); or to a thio group instead of an oxo groups. In other embodiments, a derivative of barbiturate refers to a substituted CH₂ group within the barbiturate ring (i.e. the barbiturate includes CR₁R₂ group wherein R₁ and R₂ are each independently a hydrogen, an alkyl (e.g. methyl, ethyl, isopropyl, tertbutyl), aryl (e.g., phenyl, 4-methylphenyl, naphtyl), alkenyl, alkynyl, cycloalkyl (e.g., cyclohehyl), heterocyclyl (e.g., pyridyl, imidazolyl)

As used herein, the term “alkyl” refers, in one embodiment, to a “C1 to C10 alkyl” and denotes linear and branched groups. Non-limiting examples are alkyl groups having from 1 to 6 carbon atoms (C1 to C6 alkyls), or alkyl groups having from 1 to 4 carbon atoms (C1 to C4 alkyls). Examples of alkyl groups include, but are not limited to, methyl, ethyl, n-propyl, isopropyl, n-butyl, iso-butyl, sec-butyl, tert-butyl, amyl, tert-amyl and hexyl.

The term “cycloalkyl” refers to a cyclic aliphatic C1-C10 ring. Examples of cycloalkyl groups include cyclopropane, cycloheptane, cyclohexane, cycloheptane, or cyclooctane. The cycloalkyl group may be substituted or non-substituted.

The term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. Examples of alkenyl groups include, but are not limited to, vinyl, allyl, butenyl and the like. The alkenyl group may be substituted or non-substituted.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Examples of alkynyl groups include, but are not limited to, ethynyl, propynyl and the like.

Substituted alkyl, cycloalkyl, alkenyl or alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl. C-amido, N-amido, C-carboxy, O-carboxy, sulfonamido, guanyl, guanidinyl, hydrazine, hydrazide, thiohydrazide, and amino.

The term “aryl” used herein denotes an aromatic ring system having from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubstituted or substituted through available carbon atoms with one or more groups such as halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl.

The term “heterocyclyl” refers to an aromatic or non-aromatic ring system containing from 5-14 member ring having at least one heteroatom in the ring. Non-limiting examples of suitable heteroatoms which can be included in the aromatic ring include oxygen, sulfur, phospate and nitrogen. Non-limiting examples of heterocyclic rings include pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, etc. The heteroaryl group can be unsubstituted or substituted through available carbon atoms with one or more groups such as. halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, amido, alkylamino, arylamino, heteroarylamino, dialkylamino, diarylamino, alkylarylamino, alkylheteroarylamino, arylheteroarylamino, acyl, azide, acyloxy, nitro, carboxy, carbamoyl, carboxamide, cyano, sulfonyl, sulfonylamino, sulfinyl, sulfinylamino, thiol, alkylthio, arylthio, alkylsulfonyl. In some embodiments, provided herein is a supramolecular assembly comprising a solid porous particle comprising a plurality of single-stranded polynucleotides encapsulated by melamine-cyanurate or derivative thereof. In some embodiments, provided herein is a supramolecular crystalline assembly comprising a solid porous particle comprising a plurality of single-stranded polynucleotides encapsulated by melamine-cyanurate or derivative thereof. In some embodiments, provided herein is a supramolecular assembly comprising a crystalline solid porous particle comprising a plurality of single-stranded polynucleotides encapsulated by melamine-cyanurate or derivative thereof. In some embodiments, provided herein is a supramolecular assembly comprising a solid porous particle comprising a core comprising a plurality of single-stranded polynucleotides encapsulated by melamine-cyanurate or derivative thereof. In other embodiments, the molar ratio between the melamine and the cyanurate is 1:1. In other embodiments, the single-stranded polynucleotide is ssDNA or ssRNA, or combinations thereof. In some embodiments, the single-stranded polynucleotide is ssDNA. In some embodiments, the single-stranded polynucleotide is ssRNA. In other embodiments, the single-stranded polynucleotides remain active within the particle.

In some embodiments, provided herein is a supramolecular assembly comprising a solid porous particle comprising a plurality of single-stranded polynucleotides encapsulated by melamine-barbiturate or derivative thereof. In some embodiments, provided herein is a supramolecular crystalline assembly comprising a solid porous particle comprising a plurality of single-stranded polynucleotides encapsulated by melamine-barbiturate or derivative thereof. In some embodiments, provided herein is a supramolecular assembly comprising a crystalline solid porous particle comprising a plurality of single-stranded polynucleotides encapsulated by melamine-barbiturate or derivative thereof. In some embodiments, provided herein is a supramolecular assembly comprising a solid porous particle comprising a core comprising a plurality of single-stranded polynucleotides encapsulated by melamine-barbiturate or derivative thereof. In other embodiments, the molar ratio between the melamine and the barbiturate is 1:1. In other embodiments, molar ratio of the melamine and the single stranded polynucleotide is between 0.1 to 0.000001.

In other embodiments, the single-stranded polynucleotide is ssDNA or ssRNA, or combinations thereof. In some embodiments, the single-stranded polynucleotide is ssDNA. In some embodiments, the single-stranded polynucleotide is ssRNA. In other embodiments, the single-stranded polynucleotides remain active within the particle.

In some embodiments, provided herein is a supramolecular assembly comprising a solid porous particle comprising a plurality of single-stranded polynucleotides encapsulated by non-covalent organic structure, wherein the assembly comprises a plurality of single-stranded DNA (ssDNA) or single-stranded RNA (ssRNA) polynucleotides. In other embodiments, the single-stranded polynucleotides comprise between 25 to 5000 nucleic acids. In other embodiments, the single-stranded polynucleotides comprise between 25 to 100 nucleic acids. In other embodiments, the single-stranded polynucleotides comprise between 25 to 500 nucleic acids. In other embodiments, the single-stranded polynucleotides comprise between 100 to 1000 nucleic acids. In other embodiments, the single-stranded polynucleotides comprise between 500 to 1000 nucleic acids. In other embodiments, the single-stranded polynucleotides comprise between 500 to 2000 nucleic acids. In other embodiments, the single-stranded polynucleotides comprise between 1500 to 3000 nucleic acids. In other embodiments, the single-stranded polynucleotides comprise between 2500 to 5000 nucleic acids. In other embodiments, molar ratio of the melamine and the single stranded polynucleotide is between 0.1 to 0.000001. In other embodiments, the single-stranded polynucleotides comprise one or more different single-stranded polynucleotides. In other embodiments, the single-stranded polynucleotides comprise one single-stranded polynucleotides. In other embodiments, the single-stranded polynucleotides comprise two different single-stranded polynucleotides. In other embodiments, the single-stranded polynucleotides comprise three different single-stranded polynucleotides. In other embodiments, the single-stranded polynucleotides remain active within the particle.

In some embodiments, provided herein is a supramolecular assembly comprising a solid porous particle comprising a plurality of single-stranded polynucleotides encapsulated by non-covalent organic structure, wherein, the single-stranded polynucleotides comprise one or more single-stranded polynucleotides. In other embodiments, the single-stranded polynucleotides comprise two different single-stranded polynucleotides. In other embodiments, the single-stranded polynucleotides comprise three different single-stranded polynucleotides. In other embodiments, the single-stranded polynucleotides comprise two or more different single-stranded polynucleotides. In other embodiments, the non-covalent organic structure comprises melamine-cyanurate or derivative thereof. In other embodiments, the non-covalent organic structure comprises melamine-barbiturate or derivative thereof. In other embodiments, the single-stranded polynucleotide is ssDNA or ssRNA, or combinations thereof. In some embodiments, the single-stranded polynucleotide is ssDNA. In some embodiments, the single-stranded polynucleotide is ssRNA. In other embodiments, the single-stranded polynucleotides remain active within the particle.

In some embodiments, provided herein is a supramolecular assembly comprising a solid porous particle comprising a plurality of single-stranded polynucleotides encapsulated by non-covalent organic structure and an alkaline or alkaline earth metal ions to optionally neutralize the assembly. In other embodiment, the metal ion is selected from the group consisting of: Mg²⁺, K⁺, Na⁺, Li⁺, Ca²⁺, Zn²⁺, Ce³⁺, Fe²⁺, Ni²⁺, Cu²⁺. In other embodiment, the metal ion is Mg²⁺. In other embodiments, the non-covalent organic structure comprises melamine-cyanurate or derivative thereof. In other embodiments, the non-covalent organic structure comprises melamine-barbiturate or derivative thereof. In other embodiments, the single-stranded polynucleotide is ssDNA or ssRNA, or combinations thereof. In some embodiments, the single-stranded polynucleotide is ssDNA. In some embodiments, the single-stranded polynucleotide is ssRNA. In other embodiments, the single-stranded polynucleotides remain active within the particle.

In some embodiments, the supramolecular assembly provided herein comprises a plurality of single-stranded polynucleotides encapsulated by non-covalent organic structure, wherein the single-stranded polynucleotide is covalently attached to a biomarker, dye or a fluorescent molecule.

A skilled artisan would appreciate that the term “biomarker” comprises any measurable substance in an organism whose presence is indicative of a biological state or a condition of interest. In some embodiments, the presence of a biomarker is indicative of the presence of a compound or a group of compounds of interest. In some embodiments, the concentration of a biomarker is indicative of the concentration of a compound or a group of compounds of interest.

In some embodiments, the single-stranded polynucleotide is labeled. Non limiting examples of labels include optically active dyes, such as fluorescent dyes; nanoparticles such as fluorospheres and quantum dots, rods or nanobars, and surface plasmon resonant particles (PRPs) or resonance light scattering particles (RLSs)—particles of silver or gold that scatter light.

In some embodiments, the single-stranded polynucleotide comprises a detectable moiety. In some embodiments, the single-stranded polynucleotide comprises a quencher moiety. In some embodiments, the single-stranded polynucleotide comprises a detectable moiety and a quencher moiety. In some embodiments, the detectable moiety is a dye. In some embodiments, the dye can be a fluorescent dye, e.g., a fluorophore. In some embodiments, the fluorescent dye can be a derivatized dye for attachment to the terminal 3′ carbon or terminal 5′ carbon of the single-stranded polynucleotide via a linking moiety. In some embodiments, the dye can be derivatized for attachment to the terminal 5′ carbon of the single-stranded polynucleotide via a linking moiety.

In some embodiments, the single-stranded polynucleotide comprises a fluorophore. In some embodiments, the single-stranded polynucleotide comprises one or more fluorophores. In some embodiments, the fluorophore is at the terminal 3′ carbon of the single-stranded polynucleotide. In some embodiments, the fluorophore is at the terminal 5′ carbon of the single-stranded polynucleotide.

In some embodiments, the fluorophore is an aromatic or heteroaromatic compound. In some embodiments, the fluorophore is selected from the group consisting of a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. In some embodiments, xanthene dyes comprise fluorescein and rhodamine dyes. In some embodiments, the fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R₆G), N,N,N; N′-tetramethyl-6-carboxyrhodamine (TAMRA), 6-carboxy-X-rhodamine (ROX).

In some embodiments, the single-stranded polynucleotide comprises 6-carboxyfluorescein (FAM). In some embodiments, the single-stranded polynucleotide comprises N′-tetramethyl-6-carboxyrhodamine (TAMRA).

In some embodiments, the fluorescent reporter is a naphthylamine dye that has an amino group in the alpha or beta position. In some embodiments, the naphthylamino compounds comprise 1-dimethylaminonaphthyl-5-sulfonate, 1-anilino-8-naphthalene sulfonate and 2-p-toluidinyl-6-naphthalene sulfonate, 5-(2′-aminoethyl) aminonaphthalene-1-sulfonic acid (EDANS). In some embodiments, the coumarins comprise 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes.

In some embodiments, the single-stranded polynucleotide comprises a quencher. In some embodiments, the single-stranded polynucleotide comprises one or more quenchers. As used herein, the term “quenching” may encompass the transfer of energy between the fluorophore and the quencher. In some embodiments, the single-stranded polynucleotide comprises fluorophore and a quencher. In some embodiments, the emission spectrum of the fluorophore and the absorption spectrum of the quencher overlap. In some embodiments, the fluorescent signal from the detectable moiety (i.e. fluorophore) is suppressed by the quencher. In some embodiments, cleavage of the single-stranded polynucleotide, separates the quencher moiety. The separation can enable the detectable moiety to produce a fluorescent signal. A skilled artisan would be familiar with fluorophore-quencher pairs and methods for their design and use.

In some embodiments, the quencher is a Black Hole Quencher® from Biosearch Technologies and Iowa Black®. In some embodiments, quencher is a ZEN quencher from Integrated DNA Technologies, Inc. In some embodiments, the quencher comprises a 3′-BBQ-650 (Black Berry Quencher 650), BHQ-0 (Black Hole Quencher 0, 3′), BHQ-1 (Black Hole Quencher 1, 3′), BHQ-2 (Black Hole Quencher 2, 3′), BHQ-3 (Black Hole Quencher 3, 3′), BHQ-1 (Black Hole Quencher-1, 5′), BHQ-2 (Black Hole Quencher-2, 5′), BHQ-3 (Black Hole Quencher-3, 5′), BBQ-650 NHS (Black Berry Quencher 650 NHS), BBQ-650-dT (Black Berry Quencher 650 dT), BHQ-1-NHS (Black Hole Quencher 1 NHS), TAMRA-3′ (Carboxytetramethylrhodamine). In some embodiments, the quencher is attached to the terminal 3′ carbon of the single-stranded polynucleotide. In some embodiments, the quencher is attached to the terminal 5′ carbon of the single-stranded polynucleotide.

In some embodiments, the detectable moieties and quencher moieties are covalently attached to the single-stranded polynucleotides via common reactive groups or linking moieties.

In some embodiments, the single-stranded polynucleotide comprises a spacer. In some embodiments, the single-stranded polynucleotide comprises one or more spacers. In some embodiments, the spacer comprises one or more ribose nucleotides. In some embodiments, the spacer is C3 Spacer, Hexanediol, 1′,2′-Dideoxyribose (dSpacer), PC Spacer, Spacer 9, Spacer 18. In some embodiments, the spacer at the terminal 3′ carbon of the single-stranded polynucleotide. In some embodiments, the spacer is at the terminal 5′ carbon of the single-stranded polynucleotide. In some embodiments, the spacer is located internally in the single-stranded polynucleotide. In some embodiments, the spacer is cleavable. As used herein, the term “cleavable”, may encompass the physical separation of a molecule (e.g., by cleavage, hydrolysis, or degradation) into two separate molecules.

In some embodiments, the single-stranded polynucleotide comprises a fluorescein, a quencher, and a spacer.

In some embodiments, the detectable moiety produces a non-fluorescent signal.

In other embodiments, the non-covalent organic structure comprises melamine-cyanurate or derivative thereof. In other embodiments, the non-covalent organic structure comprises melamine-barbiturate or derivative thereof. In other embodiments, the single-stranded polynucleotide is ssDNA or ssRNA, or combinations thereof. In some embodiments, the single-stranded polynucleotide is ssDNA. In some embodiments, the single-stranded polynucleotide is ssRNA. In other embodiment, the supramolecular assembly is a solid porous particle.

In some embodiments, provided herein is a supramolecular assembly comprising a solid porous particle comprising a plurality of single-stranded polynucleotides encapsulated by non-covalent organic structure, wherein, the particle size has a diameter size of between 1000000 to 100 nm. In other embodiments, the pores within the particles have a diameter of between 1000000 to 100 nm, or between 100000 to 100 nm, or between 10000 to 100 nm, or between 1000 to 100 nm.

Provided herein is a composition comprising the supramolecular assembly disclosed herein. In some embodiments, provided herein is a composition comprising a supramolecular assembly comprising a solid porous particle comprising a plurality of single-stranded polynucleotides encapsulated by non-covalent organic structure. In some embodiments, provided herein is a composition comprising a supramolecular crystalline assembly comprising a solid porous particle comprising a plurality of single-stranded polynucleotides encapsulated by non-covalent organic structure. In some embodiments, provided herein is a composition comprising a supramolecular assembly comprising a crystalline solid porous particle comprising a plurality of single-stranded polynucleotides encapsulated by non-covalent organic structure. In some embodiments, provided herein is a composition comprising a supramolecular assembly comprising a solid porous particle comprising a core comprising a plurality of single-stranded polynucleotides encapsulated by non-covalent organic structure.

In some embodiments of the composition, the supramolecular assembly protects the single-stranded polynucleotides from hydrolysis or degradation. In some embodiments of the composition, the supramolecular assembly protects the single-stranded polynucleotides from hydrolysis. In some embodiments of the composition, the supramolecular assembly protects the single-stranded polynucleotides from degradation.

In some embodiments, protecting the encapsulated single-stranded polynucleotides from degradation may encompass extending the half-life of the encapsulated single-stranded polynucleotides. In some embodiments, extending the half-life of the encapsulated single-stranded polynucleotides is relative to the half-life of single-stranded polynucleotides which are not encapsulated in the non-covalent organic structure described herein. In some embodiments, protecting the encapsulated single-stranded polynucleotides from degradation may encompass increasing the resistance to degradation by nucleases. In some embodiments, increased resistance to degradation by nucleases is relative to the degradation of single-stranded polynucleotides which are not encapsulated in the non-covalent organic structure described herein.

In some embodiments, the composition is a vaccine, diagnostic composition or analytical composition. In some embodiments, the composition is a vaccine. In some embodiments, the composition is a diagnostic composition. In some embodiments, the composition is an analytical composition.

### Methods of Preparing the Supramolecular Assembly

### Method A

In some embodiments, provided herein is a method for the preparation of the supramolecular assembly comprising a solid porous particle comprising a plurality of single-stranded polynucleotides encapsulated by melamine-cyanurate, melamine-barbiturate or derivative thereof, wherein the method comprises:
(i) forming a mixture of melamine in a polymer/matrix, optionally with an alkaline or alkaline earth metal ion, wherein the mixture is a gel or semi-solid;
(ii) forming a mixture of cyanurate or barbiturate in a polymer/matrix, optionally with an alkaline or alkaline earth metal ion, wherein the mixture is a gel or semi-solid;
(iii) forming a mixture of single-stranded polynucleotides, optionally with an alkaline or alkaline earth metal ion, wherein the mixture is a gel or semi-solid;
(iv) contacting the melamine mixture with the cyanurate or barbiturate mixture and the single-stranded mixture, such that the single-stranded polynucleotide mixture is in contact with both melamine and cyanurate or barbiturate mixtures;
(v) a reaction-diffusion of the melamine and the cyanurate, or the melamine and the barbiturate is performed, wherein the melamine and the cyanurate or barbiturate are self-assembled and encapsulate the single-stranded polynucleotides to form the solid porous particle within the polymer/matrix;
(vi) dissolve the polymer/matrix to obtain pure solid porous particle.

In other embodiments the term supramolecular assembly refers to the solid porous particle.

In some embodiments, the method for the preparation of the supramolecular assembly of Method A is prepared according to Example 1.

In some embodiments, the method for the preparation of the supramolecular assembly (or the porous particle) comprises the use of a polymer and/or a matrix as support for the reaction diffusion step. In other embodiments, the polymer/matrix comprises polysaccharide, polyacrylamide, polyethylene glycol, glycol hydrogel, gelatin hydrogel, hyaluronic acid based hydrogel. In other embodiments, the polysaccharide is agarose. In other embodiments, the strength of the polymer/matrix is between 100 g/cm² to 9,000 g/cm². In other embodiments, the strength of the polymer/matrix is between 100 g/cm² to 1,000 g/cm². In other embodiments, the strength of the polymer/matrix is between 500 g/cm² to 2,000 g/cm². In other embodiments, the strength of the polymer/matrix is between 500 g/cm² to 4,000 g/cm². In other embodiments, the strength of the polymer/matrix is between 1000 g/cm² to 2,000 g/cm². In other embodiments, the strength of the polymer/matrix is between 1000 g/cm² to 3,000 g/cm². In other embodiments, the strength of the polymer/matrix is between 2000 g/cm² to 5,000 g/cm². In other embodiments, the strength of the polymer/matrix is between 4000 g/cm² to 9,000 g/cm². In other embodiments, the polymer/matrix forms a gel following heating to a temperature of 100° C. In other embodiments, steps (i), (ii) and (iii), of the method described for the preparation of the supramolecular assembly, further comprises a step of heating, in order to obtain a gel or semi-solid form.

In some embodiments, the method for the preparation of the supramolecular assembly (or the porous particle) comprises forming a mixture of single-stranded polynucleotides, optionally with an alkaline or alkaline earth metal ion, wherein the mixture is a gel or semi-solid (step (iii)). In other embodiments, in other embodiments, the single-stranded polynucleotide is ssDNA or ssRNA, or combinations thereof. In some embodiments, the single-stranded polynucleotide is ssDNA. In some embodiments, the single-stranded polynucleotide is ssRNA. In other embodiments, the single-stranded polynucleotide is covalently attached to a dye or to a fluorescent probe.

In some embodiments, the method for the preparation of the supramolecular assembly (or the porous particle) comprises contacting the melamine mixture with the cyanurate or barbiturate mixture and the single-stranded mixture, such that the single-stranded polynucleotide mixture is in contact with both melamine and cyanurate or barbiturate mixtures (step (iv); wherein each of the mixtures are at pH between 3-12.

In some embodiments, the method for the preparation of the supramolecular assembly (or the porous particle) comprises a reaction-diffusion of the melamine and the cyanurate, or the melamine and the barbiturate, wherein the melamine and the cyanurate or barbiturate are self-assembled and encapsulate the single-stranded polynucleotides to form the solid porous particle within the polymer/matrix (step (v); wherein the reaction diffusion occurs at pH of between 3-10 and temperature of between 40-80° C. In other embodiments, the reaction diffusion is performed in a period of between 10 min to 2 days.

In other embodiments, the concentration of the melamine within the polymer/matrix is between 1.0-50 mM.

In other embodiments, the concentration of the cyanurate or barbiturate within the polymer/matrix is between 0.2-20 mM. In other embodiments, the concentration of the cyanurate or barbiturate within the polymer/matrix is between 0.2-2 mM. In other embodiments, the concentration of the cyanurate or barbiturate within the polymer/matrix is between 0.5-5 mM. In other embodiments, the concentration of the cyanurate or barbiturate within the polymer/matrix is between 1-10 mM. In other embodiments, the concentration of the cyanurate or barbiturate within the polymer/matrix is between 5-20 mM.

In other embodiments, the concentration of the single-stranded polynucleotides within the polymer/matrix is between 1-1000 nM. In other embodiments, the concentration of the single-stranded polynucleotides within the polymer/matrix is between 1-50 nM. In other embodiments, the concentration of the single-stranded polynucleotides within the polymer/matrix is between 1-100 nM. In other embodiments, the concentration of the single-stranded polynucleotides within the polymer/matrix is between 100-1000 nM. In other embodiments, the concentration of the single-stranded polynucleotides within the polymer/matrix is between 50-500 nM.

In other embodiments, the concentration of the metal ion within the polymer/matrix is between 0 mM to 500 mM. In other embodiments, the concentration of the metal ion within the polymer/matrix is between 0 mM to 100 mM. In other embodiments, the concentration of the metal ion within the polymer/matrix is between 0 mM to 200 mM. In other embodiments, the concentration of the metal ion within the polymer/matrix is between 0 mM to 300 mM. In other embodiments, the concentration of the metal ion within the polymer/matrix is between 0 mM to 400 mM. In other embodiments, the concentration of the metal ion within the polymer/matrix is between 10 mM to 100 mM. In other embodiments, the concentration of the metal ion within the polymer/matrix is between 50 mM to 200 mM. In other embodiments, the single-stranded polynucleotide is ssDNA or ssRNA, or combinations thereof. In other embodiments, the single-stranded polynucleotides remain active within the particle.

In some embodiments, molar ratio of the melamine and the single stranded polynucleotide is between 0.1 to 0.000001. In other embodiments, the molar ratio of the melamine and the single stranded polynucleotide is between 0.1 and 0.000001, or 0.1 and 0.00001, or 0.1 and 0.0001, or 0.1 and 0.01, or 0.1 and 0.1.

### Method B

In some embodiments, provided herein is a method for the preparation of the supramolecular assembly comprising a solid porous particle comprising a plurality of single-stranded polynucleotides encapsulated by melamine-cyanurate, melamine-barbiturate or derivative thereof, wherein the method comprises:
(a) prepare a Solution 1 comprising of cyanurate (cyanuric acid) or barbiturate;
(b) prepare Solution 2 comprising melamine at the same concentration of cyanurate;
(c) dissolve a single stranded polynucleotide in Solution 1, or Solution 2, or prepare Solution 3 comprising a single stranded polynucleotide and further add Solution 3 to Solution 1 or Solution 2;
(d) optionally add a metal ion salt to Solution 1 or to Solution 2;
(e) mix equal volumes of Solution 1 and Solution 2 to obtain a reaction mixture; and
(f) precipitate a solid porous particle.

In some embodiments, provided herein is a method (Method B) for the preparation of the supramolecular assembly, wherein the method comprises preparing Solution 1 comprising cyanurate or barbiturate at a concentration of between 1 mM to 20 mM. In other embodiments, the solution is an aqueous solution. In other embodiments, the method comprises preparing Solution 1 comprising cyanurate or barbiturate at a concentration of between 1 mM to 15 mM. In other embodiments, the method comprises preparing Solution 1 comprising cyanurate or barbiturate at a concentration of between 10 mM to 15 mM. In other embodiments, the method comprises preparing Solution 1 comprising cyanurate or barbiturate at a concentration of between 10 mM to 20 mM. In other embodiments, the method comprises preparing Solution 1 comprising cyanurate or barbiturate at a concentration of 2, 4, 6, 8, 10, 11, 12, 13, 14, 15, 16, 17, 18 19 or 20 mM.

In some embodiments, provided herein is a method (Method B) for the preparation of the supramolecular assembly, wherein the method comprises preparing Solution 2 comprising melamine at a concentration of between 1 mM to 20 mM. In other embodiments, the solution is an aqueous solution. In other embodiments, the pH of Solution 1 is between 4 to 8. In other embodiments, the method comprises preparing Solution 2 comprising melamine at a concentration of between 1 mM to 15 mM. In other embodiments, the method comprises preparing Solution 2 comprising melamine at a concentration of between 10 mM to 15 mM. In other embodiments, the method comprises preparing Solution 2 comprising melamine at a concentration of between 10 mM to 20 mM. In other embodiments, the method comprises preparing Solution 2 comprising melamine at a concentration of 2, 4, 6, 8, 10, 11, 12, 13, 14, 15, 16, 17, 18 19 or 20 mM.

In some embodiments, provided herein is a method (Method B) for the preparation of the supramolecular assembly, wherein the method comprises dissolving a single stranded polynucleotide in Solution 1 or Solution 2. In other embodiments, the pH of Solution 2 is between 4 to 8. In other embodiments, the single stranded polynucleotide is ssDNA or ssRNA.

In some embodiments, provided herein is a method (Method B) for the preparation of the supramolecular assembly, wherein the method comprises preparing Solution 3 comprising a single stranded polynucleotide and further adding Solution 3 to Solution 1 or Solution 2. In other embodiments, the single stranded polynucleotide is ssDNA or ssRNA.

In some embodiments, provided herein is a method (Method B) for the preparation of the supramolecular assembly, wherein the method comprises optionally adding a metal ion salt to Solution 1 or to Solution 2. In other embodiments, metal ion salt is not added. In other embodiments, metal ion salt is Mg2+.

In some embodiments, provided herein is a method (Method B) for the preparation of the supramolecular assembly, wherein the method comprises mixing equal volumes of Solution 1 and Solution 2 to obtain a reaction mixture. In some embodiments, no metal ion salt is added. In some embodiments, the concentration of the metal ion salt within the reaction mixture is between 0.1 mM to 100 mM. In other embodiments, the concentration of the metal ion within the reaction mixture is between 0.1 mM to 200 mM. In other embodiments, the concentration of the metal ion within the reaction mixture is between 0.1 mM to 300 mM. In other embodiments, the concentration of the metal ion within the reaction mixture is between 0.1 mM to 400 mM. In other embodiments, the concentration of the metal ion within the reaction mixture is between 10 mM to 100 mM. In other embodiments, the concentration of the metal ion within the reaction mixture is between 100 mM to 500 mM. In other embodiments, the concentration of the metal ion within the reaction mixture is between 50 mM to 200 mM.

In other embodiments, the concentration of the single stranded polynucleotide within the reaction mixture is between 1 pM-500 nM. In other embodiments, the concentration of the single-stranded polynucleotide is between 1 pM-1 nM. In other embodiments, the concentration of the single-stranded polynucleotide is between 1 pM-500 pM. In other embodiments, the concentration of the single-stranded polynucleotide is between 1 pM-100 pM. In other embodiments, the concentration of the single-stranded polynucleotide is between 1 nM-100 nM. In other embodiments, the concentration of the single-stranded polynucleotide is between 100 nM-500 nM.

In some embodiments, provided herein is a method (Method B) for the preparation of the supramolecular assembly, wherein the method comprises mixing equal volumes of Solution 1 and Solution 2 to obtain a reaction mixture at a temperature of between 2° and 40° C. In other embodiments, the method comprises mixing equal volumes of Solution 1 and Solution 2 to obtain a reaction mixture at pH of between 4 and 8. In other embodiments, the pH of the reaction mixture is between 4-6. In other embodiments, the pH is between 6-8. In other embodiments, the pH is 4, 5, 6, 7 or 8. In other embodiments, the reaction mixture is at a temperature of 20° C., 35° C., 30° C., 40° C. In other embodiments, the reaction mixture is at room temperature. In other embodiments, the reaction mixture is at ambient temperature.

In some embodiments, provided herein is a method (Method B) for the preparation of the supramolecular assembly, wherein the method comprises precipitating a solid porous particle. In other embodiments, the particle is precipitated by mixing Solutions 1 and 2.

In some embodiments, the methods for the preparation of supramolecular assembly (Method A and Method B) comprise a method for storing single-stranded polynucleotides. In other embodiments, the single-stranded polynucleotide is ssDNA or ssRNA, or combinations thereof.

### Methods of Releasing the Polynucleotide

In some embodiments, the single-stranded polynucleotides within the solid porous particle are released upon demand or need by applying a pH of between 3-10 and/or a temperature of between 40-80° C. In other embodiments, the single-stranded polynucleotide is ssDNA or ssRNA, or combinations thereof. In other embodiments, the single-stranded polynucleotides within the solid porous particle are released upon demand or need by applying a pH of 3, 4, 5, 6, 7, 8, 9 or 10.

In some embodiments, provided herein is a supramolecular assembly comprising a solid porous particle comprising a plurality of single-stranded polynucleotides encapsulated by non-covalent organic structure, wherein the single-stranded polynucleotides within the solid porous particle are released upon demand or need by applying a pH of between 3-10 and/or a temperature of between 40-80° C. In other embodiments, the single-stranded polynucleotide is ssDNA or ssRNA, or combinations thereof. In other embodiments, the single-stranded polynucleotides within the solid porous particle are released upon demand or need by applying a pH of 3, 4, 5, 6, 7, 8, 9 or 10.

### Methods of Use for Supramolecular Assembly

### Detecting the Presence of a Nucleotide Sequence

In some embodiments, provided herein is a method of detecting the presence of a nucleotide sequence in a sample, the method comprising: obtaining a sample; contacting the sample with a supramolecular assembly comprising a solid porous particle comprising a plurality of single-stranded polynucleotides encapsulated by non-covalent organic structure; adjusting the pH of the solution for dissolution of particles; and detecting the presence of a nucleotide sequence.

In some embodiments of the methods described herein, adjusting the pH of the solution for dissolution of particles comprises applying a pH of between 3-10 and/or a temperature of between 40-80° C. In some embodiments of the methods described herein, adjusting the pH of the solution for dissolution of particles comprises applying a pH of between 3-10. In some embodiments, the pH for dissolution of particles is 3, 4, 5, 6, 7, 8, 9 or 10. In some embodiments, the pH for dissolution of particles is 3. In some embodiments, the pH for dissolution of particles is 4. In some embodiments, the pH for dissolution of particles is 5. In some embodiments, the pH for dissolution of particles is 6. In some embodiments, the pH for dissolution of particles is 7. In some embodiments, the pH for dissolution of particles is 8. In some embodiments, the pH for dissolution of particles is 9. In some embodiments, the pH for dissolution of particles is 10.

In some embodiments of the method of detecting the presence of a nucleotide sequence in a sample, the method further comprises the step of concentrating the contacted solution.

In some embodiments, the methods herein are used to diagnose, prognose, monitor or observe cancers or other diseases. In some embodiments, the methods herein are used for non-invasive prenatal testing. In other embodiments, the methods are used to diagnose, prognose, monitor or observe cancers or other diseases in an unborn subject in amniotic fluid.

In some embodiments of a method of detecting the presence of a nucleotide sequence in a sample, the sample is contacted with a supramolecular assembly comprising a solid porous particle comprising a plurality of single-stranded polynucleotides encapsulated by non-covalent organic structure, as described herein in detail. In some embodiments of a method of detecting the presence of a nucleotide sequence in a sample, the sample is contacted with a composition comprising the supramolecular assembly comprising a solid porous particle comprising a plurality of single-stranded polynucleotides encapsulated by non-covalent organic structure, as described herein in detail.

In some embodiments, the supramolecular assembly comprises a solid porous particle comprising a plurality of single-stranded polynucleotides encapsulated by non-covalent organic structure, wherein the non-covalent organic structure comprises melamine and cyanurate or melamine and barbiturate or derivative thereof. In other embodiments the non-covalent organic structure is solid. In other embodiments the non-covalent organic structure is crystalline.

In some embodiments, the non-covalent organic structure comprises melamine-cyanurate or a derivative thereof. In other embodiments, the molar ratio between the melamine and the cyanurate is 1:1.

In some embodiments, the non-covalent organic structure comprises melamine-barbiturate or a derivative thereof. In other embodiments, the molar ratio between the melamine and the barbiturate is 1:1.

In some embodiments, the single-stranded polynucleotide is ssDNA or ssRNA, or combinations thereof. In some embodiments, the single-stranded polynucleotide is ssDNA. In some embodiments, the single-stranded polynucleotide is ssRNA. In other embodiments, the single-stranded polynucleotides remain active within the particle.

In some embodiments, methods of detecting the presence of a nucleotide sequence involve the use of detectable probes that are designed to bind to and enable detection of reaction products. In some embodiments, methods of detecting the presence of a nucleotide sequence, the detectable probe includes a detectable moiety and can further include a quencher moiety that inhibits the detectable moiety from emitting a detectable signal (such as a fluorescent moiety).

In some embodiments, methods of detecting the presence of a nucleotide sequence, the single-stranded polynucleotide comprises a reporter probe. In some embodiments, the reporter probe is designed to produce a detectable signal indicating the presence of the nucleotide sequence of interest.

In some embodiments, methods of detecting the presence of a nucleotide sequence utilize intercalating dyes that fluoresce in the presence of double stranded DNA (dsDNA), such as molecular beacons.

In some embodiments, the single-stranded polynucleotide is labeled with a molecular beacon. An artisan would appreciate that a “molecular beacon” may encompass oligonucleotide hybridization probes that can report the presence of specific nucleic acids in homogenous solutions. Molecular beacons are hairpin-shaped molecules with an internally quenched fluorophore whose fluorescence is restored when they bind to a target nucleic acid sequence. In some embodiments, molecular beacons can be used as probes, where there is no fluorescence when the polynucleotide is in a single strand molecule, and only when a double strand is formed the stem unwinds and releases the fluorophore from quenching.

In some embodiments, the single-stranded polynucleotide comprises a fluorophore. In some embodiments, the single-stranded polynucleotide comprises one or more fluorophores. In some embodiments, the fluorophore is at the terminal 3′ carbon of the single-stranded polynucleotide. In some embodiments, the fluorophore is at the terminal 5′ carbon of the single-stranded polynucleotide.

In some embodiments, the fluorophore is an aromatic or heteroaromatic compound. In some embodiments, the fluorophore is selected from the group consisting of a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. In some embodiments, xanthene dyes comprise fluorescein and rhodamine dyes. In some embodiments, the fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R₆G), N,N,N; N′-tetramethyl-6-carboxyrhodamine (TAMRA), 6-carboxy-X-rhodamine (ROX).

In some embodiments, the single-stranded polynucleotide comprises a quencher. In some embodiments, the single-stranded polynucleotide comprises one or more quenchers. In some embodiments, the quencher is a Black Hole Quencher® from Biosearch Technologies and Iowa Black®. In some embodiments, quencher is a ZEN quencher from Integrated DNA Technologies, Inc. In some embodiments, the quencher comprises 3′-BBQ-650 (Black Berry Quencher 650), BHQ-0 (Black Hole Quencher 0, 3′), BHQ-1 (Black Hole Quencher 1, 3′), BHQ-2 (Black Hole Quencher 2, 3′), BHQ-3 (Black Hole Quencher 3, 3′), BHQ-1 (Black Hole Quencher-1, 5′), BHQ-2 (Black Hole Quencher-2, 5′), BHQ-3 (Black Hole Quencher-3, 5′), BBQ-650 NHS (Black Berry Quencher 650 NHS), BBQ-650-dT (Black Berry Quencher 650 dT), BHQ-1-NHS (Black Hole Quencher 1 NHS), TAMRA-3′ (Carboxytetramethylrhodamine). In some embodiments, the quencher is attached to the terminal 3′ carbon of the single-stranded polynucleotide. In some embodiments, the quencher is attached to the terminal 5′ carbon of the single-stranded polynucleotide.

In some embodiments, the single-stranded polynucleotide comprises a spacer. In some embodiments, the single-stranded polynucleotide comprises one or more spacers. In some embodiments, the spacer comprises one or more ribose nucleotides. In some embodiments, the spacer is C3 Spacer, Hexanediol, 1′,2′-Dideoxyribose (dSpacer), PC Spacer, Spacer 9, Spacer 18. In some embodiments, the spacer at the terminal 3′ carbon of the single-stranded polynucleotide. In some embodiments, the spacer is at the terminal 5′ carbon of the single-stranded polynucleotide. In some embodiments, the spacer is located internally in the single-stranded polynucleotide. In some embodiments, the spacer is cleavable.

In some embodiments, the single-stranded polynucleotide comprises a fluorescein and a quencher. In some embodiments, detecting the presence of a nucleotide sequence comprises by detecting fluorescence. In some embodiments, detecting the presence of a nucleotide sequence is achieved following separation (for example, following cleavage) of the fluorescein and quencher in the single-stranded polynucleotide. Separation of the fluorescein and quencher can occur by cleavage of the single-stranded polynucleotide.

In some embodiments, the single-stranded polynucleotide comprises a fluorescein, a quencher, and a spacer.

In some embodiments, the sample is a liquid sample. In some embodiments, the sample is obtained from a bodily fluid or a water source. In some embodiments, the sample is obtained from a water source. In some embodiments, the sample is obtained from a bodily fluid. In some embodiments, the bodily fluid is selected from the group consisting of blood, serum, plasma, saliva, sputum, feces, urine, and spinal fluid. In some embodiments, the sample is derived from an amniotic fluid sample. In some embodiments, the sample is derived from an umbilical cord sample. In some embodiments, the bodily fluid is blood. In some embodiments, the bodily fluid is serum. In some embodiments, the bodily fluid is plasma. In some embodiments, the bodily fluid is saliva. In some embodiments, the bodily fluid is sputum. In some embodiments, the bodily fluid is feces. In some embodiments, the bodily fluid is urine. In some embodiments, the bodily fluid is spinal fluid. In some embodiments, the sample is derived from a tissue (e.g., a tissue homogenate), a biopsy, and/or a tumor.

In some embodiments, the nucleotide sequence in a sample to be detected can be any nucleotide sequence of interest. In some embodiments, the nucleotide sequence is derived from one or more of an animal, a human, a plant, a cultured cell, a microorganism, a virus, a bacterium, or a pathogen. In some embodiments, the nucleotide sequence is derived from a pathogen. In some embodiments, the pathogen is a bacterial, fungal or viral pathogen. In some embodiments, the pathogen is a bacterial pathogen. In some embodiments, the pathogen is a fungal pathogen. In some embodiments, the pathogen is a viral pathogen.

In some embodiments, the nucleotide sequence is selected from the group consisting of double-stranded (ds) nucleic acids, single stranded (ss) nucleic acids, DNA, RNA, cDNA, mRNA, CRNA, IRNA, ribosomal RNA, dsDNA, ssDNA, miRNA, siRNA, circulating nucleic acids, circulating cell-free nucleic acids, circulating DNA, circulating RNA, cell-free nucleic acids, cell-free DNA, cell-free RNA, circulating cell-free DNA, cell-free dsDNA, cell-free ssDNA, circulating cell-free RNA, genomic DNA, exosomes, cell-free pathogen nucleic acids, circulating pathogen nucleic acids, mitochondrial nucleic acids, non-mitochondrial nucleic acids, nuclear DNA, nuclear RNA, chromosomal DNA, circulating tumor DNA, circulating tumor RNA, circular nucleic acids, circular DNA, circular RNA, circular single-stranded DNA, circular double-stranded DNA, plasmids, bacterial nucleic acids, fungal nucleic acids, parasite nucleic acids, viral nucleic acids, cell-free bacterial nucleic acids, cell-free fungal nucleic acids, cell-free parasite nucleic acids, viral particle-associated nucleic acids, viral-particle free nucleic acids or any combination thereof. In some embodiments, the nucleotide sequence is DNA. In some embodiments, the nucleotide sequence is ssDNA. In some embodiments, the nucleotide sequence is RNA. In some embodiments, the nucleotide sequence is cDNA. In some embodiments, the nucleotide sequence is siRNA. In some embodiments, the nucleotide sequence is mitochondrial DNA. In some embodiments, the nucleotide sequence is mRNA. In some embodiments, the nucleotide sequence is selected from one or more of DNA, RNA, cDNA, mRNA, CRNA, IRNA, ribosomal RNA, dsDNA, ssDNA, miRNA, siRNA, and mitochondrial DNA.

### Use for Gene Suppression/Expression

In some embodiments, provided herein is a method of gene suppression in a cell, the method comprising: contacting the cell with a supramolecular assembly comprising a solid porous particle comprising a plurality of single-stranded polynucleotides encapsulated by non-covalent organic structure.

In one embodiment, the term “gene suppression” may encompass reduction or suppression of expression (i.e. down-regulation) of a target protein in a host cell as the result of transcription of a single-stranded polynucleotide. In some embodiments, the single-stranded polynucleotide comprises an RNA having a gene silencing effect. In some embodiments, the single-stranded polynucleotide comprises antisense RNA. In some embodiments, the single-stranded polynucleotide comprises interfering RNA (RNAi). In some embodiments, the single-stranded polynucleotide comprises siRNA.

In one embodiment, the down-regulation is achieved by antisense RNA. In another embodiment, the down-regulation is achieved by ribozyme technology, which, in one embodiment, works at the RNA translational level and involves making catalytic RNA molecules which bind to and cleave the mRNA of interest. Both of these were found effective in regulating protein levels in plants. In another embodiment, the down-regulation is achieved by co-suppression.

In some embodiments, the down-regulation is achieved by DNAzyme molecules. In some embodiments, the single-stranded polynucleotide comprises a DNAzyme.

In some embodiments, the DNAzyme is capable of down-regulating the expression of a given gene. In some embodiments, the DNAzyme is capable of specifically cleaving an mRNA transcript or a DNA sequence of said gene.

An artisan would appreciate that DNAzymes are single-stranded polynucleotides that are capable of cleaving both single- and double-stranded target sequences. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine: pyrimidine junctions. An artisan would be familiar with the methods of designing synthetic, engineered DNAzymes recognizing single- and double-stranded target cleavage sites.

In some embodiments, provided herein is a method of expressing a gene in a cell, the method comprising: contacting the cell with a supramolecular assembly comprising a solid porous particle comprising a plurality of single-stranded polynucleotides encapsulated by non-covalent organic structure.

In some embodiments, the single-stranded polynucleotide comprises a recombinant polynucleotide. In one embodiment, single-stranded polynucleotides described herein comprise recombinant polynucleotides providing for expression of mRNA encoding a polypeptide. In another embodiment, single-stranded polynucleotides described herein comprise recombinant polynucleotides providing for expression of mRNA.

In some embodiments, contacting the cell with a supramolecular assembly comprises delivering the supramolecular assembly described herein to a cell. In some embodiments, the supramolecular assembly described herein is introduced to a cell. In some embodiments, the cell is a plant cell.

In some embodiments, the plant cell is a germ line cell. The term “germ line cell” refers to a cell in the plant organism which can trace their cell lineage to either the male or female reproductive cell of the plant. In some embodiments, the plant cell is a somatic cell. The term “somatic cell” is a cell which gives rise to leaves, roots and vascular elements

In some embodiments, the plant cell is regenerated to obtain a whole plant. The term “regeneration” as used herein means growing a whole plant from a plant cell, a group of plant cells, a plant part (including seeds), or a plant piece (e.g., from a protoplast, callus, or tissue part). The methods provided herein may be used for plant genetic engineering.

An artisan would be familiar with the various methods of plant transformation for introducing genetic material into a plant cell. In some embodiments, the supramolecular assembly is transformed or delivered to a cell by particle bombardment or microinjection. Particle bombardment includes biolistic transfection or microparticle-mediated gene transfer, which refers to a physical delivery method for transferring a microparticle or nanoparticle comprising a nucleic acid or a genetic construct of interest into a target cell or tissue. The transformation via particle bombardment uses a microprojectile of metal covered with the construct of interest, which is then shot onto the target cells using an equipment known as “gene gun” at high velocity, enabling penetration of the cell wall of a target tissue.

Various embodiments and aspects of this invention as delineated hereinabove and as claimed in the claims section below find experimental support in the following examples.

### EXAMPLES

### Materials and Methods

Reagents and materials. Melamine (M, C3H6N6) and cyanuric acid (CA, C3H3N303) were purchased from Sigma-Aldrich. TAMRA-DNA, T-sub, Dzle used in this study were synthesized by DNK-sintez, Russia. TAMRA-DNA was purified by polyacrylamide gel electrophoresis (PAGE) and sublimated by DNK-syntez company (Russia, Moscow).

Oligonucleotides' preparation. The delivered sublimated TAMRA-DNA (TAMRA-cagtcgactcgccgattagacgaaca SEQ ID NO: 11) was diluted with Nuclease free-water (Invitrogen, USA) to a concentration of 100 μM.

The delivered sublimated T-sub (TAMRA-GTTTCCTCguCCCTGG-BHQ2 SEQ ID NO: 1) was diluted with Nuclease free-water (Invitrogen, USA) to a concentration of 100 μM.

The delivered sublimated Dz1 (CCAGGGAGGCTAGCTACAACGAGAGGAAAC SEQ ID NO: 2) was diluted with Nuclease free-water (Invitrogen, USA) to a concentration of 100 μM.

TABLE 2
showing exemplary ssDNAs and ssRNA:

| SEQ ID | | |
|---|---|---|
| NO: | Sequence | Description of sequence |
| 1 | TAMRA-gtttcctc(rGrU)ccctgg-BHQ2 | T_sub DNA having |
| | | Modifications: TAMRA = 5- |
| | | Carboxytetramethylrhodamine (fluorophore) |
| | | at 5′ |
| | | BHQ2 = Black Hole Quencher-2 at 3′ |
| | | (rGrU) = ribose spacer (RNA) nucleotides |
| 2 | ccagggaggctagctacaacgagaggaaac | Dz1e DNAzyme |
| 3 | ccagggaggctagctacaacgagaggaaacac(rGrU) | IDz1 DNA having |
| | catcagggaaaagcctccctaaa | Modification: (rGrU) = ribose spacer (RNA) |
| | | nucleotides |
| 4 | cctgatgaggctagctacaacgagtgtttcc | Dz2 DNAzyme |
| 5 | aaccacacaacacaacgagtgtttcc | Dza2 DNAzyme |
| 6 | cctgatgaggctagctctactacctca | Dzb2 DNAzyme |
| 7 | tgaggtagtaggttgtgtggtt | let-7b DNAzyme |
| 8 | cacacgtgttctgttcgtcta | T1 DNAzyme |
| 9 | cctgatgaggctagctctactacctca(iSp9)tagacga | T2 Modification: Spacer 9 internally |
| | acgaaacacgtgtg(iSp9)aaccacacaacacaacga | |
| | gtgtttcc | |
| 10 | 6-FAM-cagucgacucgccgauuagacgaaca | ssRNA with Modification: 6-FAM |
| | | (Fluorescein) at 5′ |
| 11 | TAMRA-cagtcgactcgccgattagacgaaca | TAMRA-DNA |

Fluorescence spectrophotometry assay. The intensity of fluorescence of TAMRA-modified ssDNA was measured by a fluorescence spectrophotometer Aglient Cary 60 (Aglient Technologies, USA). 100 nM TAMRA-DNA was mixed with an analyzing buffer (150 mM KCl, 50 HEPES, 50 MgCl2, 15 mM NaCl, deionized water). The mixture was loaded into a quartz micro cell (Starna GmbH, Germany). A set up for laser wavelengths: excitation=545 nm; emission=690 nm.

Fluorescence Images. Fluorescence microscopy images were obtained using Leica DMI8 microscope equipped with RHOD filter. Images were made in bright field (BF) and RHOD channels. Acquisition parameters in BF mode: intensity=100, gain=1, aperture=16. Acquisition parameters in RHOD channel: FIM=100, gain=1, Il-Fl=4.

Fluorescence during the assembly. To study changes in fluorescence of a particle ImageJ program package was used. Fluorescence intensity profiles of particles were extracted. Profiles plot was made using OriginLab program package. For the average intensity profiles several particles were analyzed, and resulting profiles were averaged using OriginLab.

Fluorescence during the disassembly. The procedure was the same as for assembly. The values of fluorescence intensity in the center and at the edge of the particle and background were measured pointwise.

Size and the number of particles. To count particles and measure their size several images of each sample were analyzed using ImageJ program package. Images were from the same gel section. The circularity parameter was set 0.7-1.0.

Scanning electron microscopy (SEM) and energy-dispersive X-Ray analysis (EDX). SEM images were obtained using a Hitachi S-3400N (Japan) equipped with Oxford X-Max 20 (UK) EDX spectrometer. Acquisition conditions were as follows: 20 kV accelerating voltage, 1 nA beam current, 60 sec per spectrum in point mode, 10 kV; 1 nA and 90 min acquisition time for mapping. The samples were coated with 2 nm of carbon.

PAGE electrophoresis. Denaturing PAGE was performed using Mini-PROTEAN vertical electrophoresis system (BIORAD) at 80V (constant voltage) for 2 hours. Gel contained 15% polyacrylamide (40% AA/BA), 7M Urea, 1×TBE, 10% ammonium persulfate (ACS) (100 μl/10 ml of the gel solution) and TEMED (10 μl/10 ml of the gel solution). 5 μl of 0.5 M NaOH was added to 20 μl water solution with purified particles and left for 5 minutes. After 5 μl of 0.5 M HCl to neutralize NaOH and 20 μl of a loading buffer (8M Urea, 15% 2×TBE) were added. Each lane contained 20 μl of the final solution.

### Molecular Dynamics (MD) Simulation Details

The classical molecular dynamics (MD) approach is an acceptable method to study the M-CA nucleation processes around ssDNA as well as the influence of cations from atomistic level of description. This method is massively used to study the interactions of simple ions and polycations with DNA. It was previously demonstrated that Mg²⁺ make only simultaneous contacts with multiple competing electronegative sites of DNA that last about 1 ns.

### Example 1

### Particle Formation

Preparation of agar gels. 1 wt % agar gels containing 13 mM melamine (M), 100 nM ssDNA and 13 mM cyanurate (CA) were prepared in the presence of TBE buffer and required MgCl₂ concentration. The final TBE concentration after dilution during gel preparation is 1×. The aqueous solution of MgCl₂ (1M) was diluted in 5, 20 and 100 times to get 200 mM, 50 mM and 10 mM correspondingly after gel preparation. Gels with 500 mM MgCl₂ were prepared using MgCl₂·6H₂O. The mixtures were heated to 100° C. to dissolve agar. In contrast to M and CA, ssDNA solution is added after gel cooling to ˜ 40° C. not before components mixing. The procedure for preparing T-sub and Dz1 containing gels is the same.

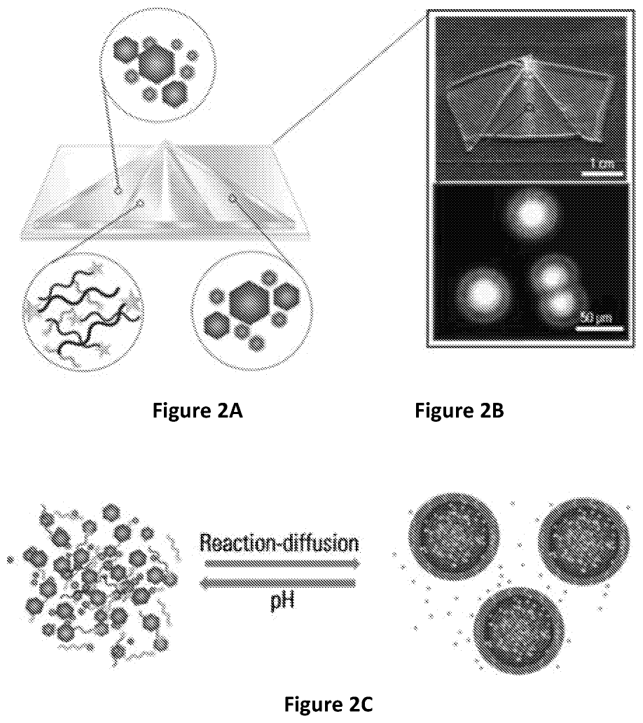

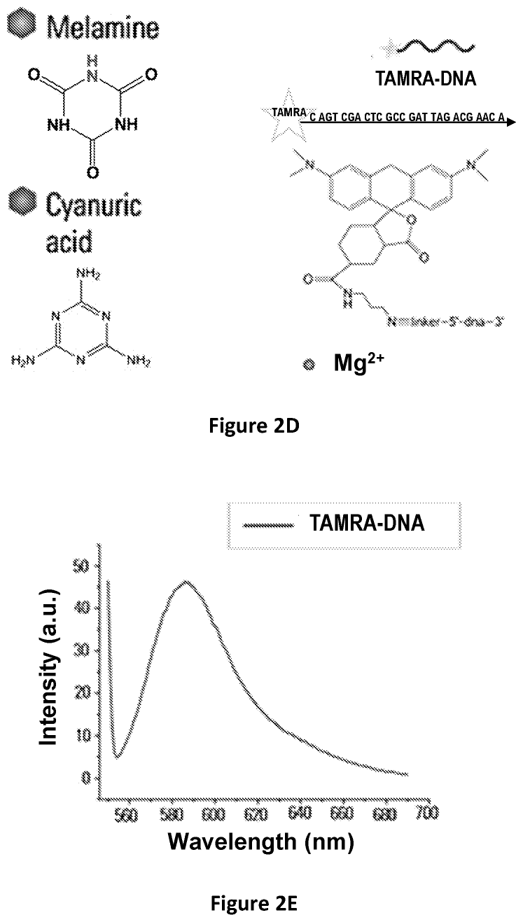

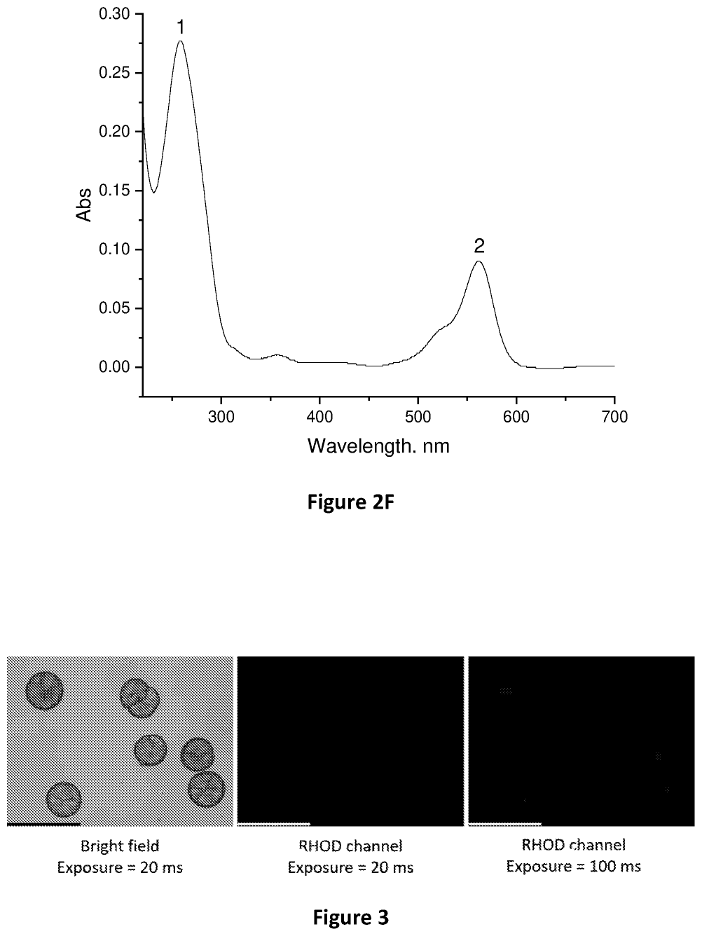

Formation of particles. Hot gels were poured in glass cuvettes and left till complete gelation. Melamine, ssDNA and cyanuric acid gels with required size and form were completed and combined in the reaction-diffusion system on glass slide (FIG. 2A-2B). Particles began to form almost a few minutes later. Samples were left until complete particle formation with provided extra humidity to prevent gel drying.

Purification. Gel with formed particles was put into centrifuge tube filled with dH₂O. Then, it was heated using water to dissolve gel. Then, samples were centrifuged, a supernatant was removed and dH₂O was added to a precipitate. Heating and centrifuging were repeated twice to get water solution with pure precipitate.

The reaction-diffusion process was used for particle formation. The schematic experiment is shown in FIG. 2A. Agar gels of three initial reactants were fabricated in special reservoirs and cut as triangles. The reaction-diffusion system consisted of three connected segments where the encapsulated reactant was in the middle. M and CA diffusing fronts cross at different times in top and the bottom of ssDNA gel triangle, as the space between M and CA was different. The particles form strictly in between, and with the increase of interspace the particles form slower. Thus, it was easy to observe particles formation from beginning to the end due to the triangular cut of agar gels. Local formation of white particles were observed at the crossing of diffusing reactants fronts and surprisingly bright luminescence of the particles were seen (FIG. 2B). Reaction-diffusion system was reversible and with pH change the particles disassemble with release of all constituents, as shown in FIG. 2C. The system consisting of M, CA, modified ssDNA and Mg²⁺ (FIG. 2D) was studied. ssDNA was modified with TAMRA fluorophore to monitor the encapsulation process (FIG. 2E, FIG. 2F). M-CA particles did not show significant luminescence in red fluorescence channel (excitation 546 nm, emission 585 nm, RHOD) (FIG. 3) without ssDNA.

### Example 2

### Time Dependent Study of the Assembly of the M-CA

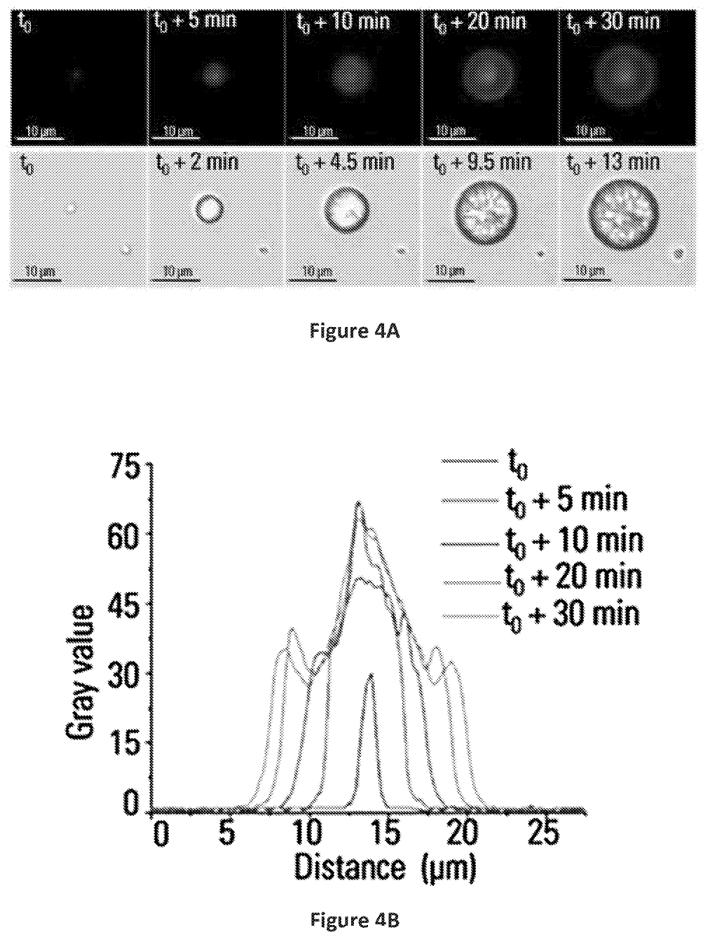

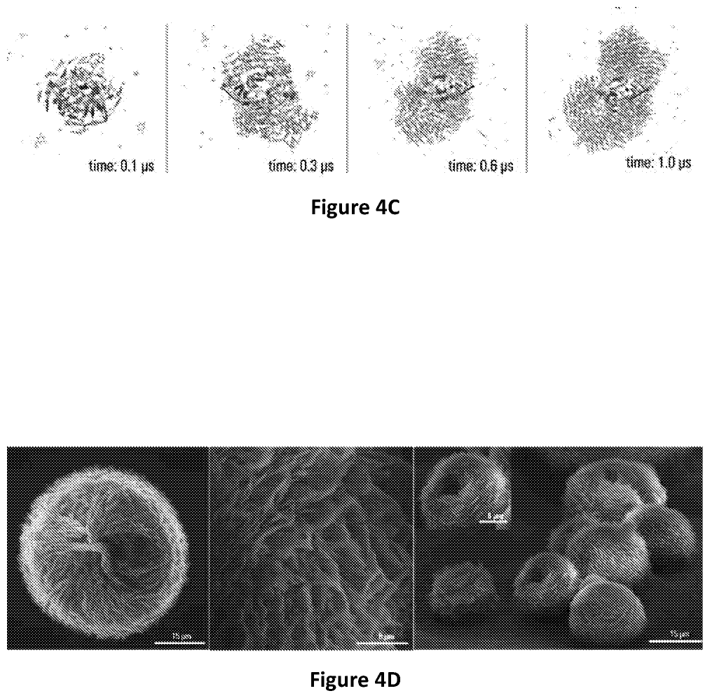

To investigate the details of the assembly dynamics, time-dependent experiments were performed (FIG. 4A, with sequential imaging of an assembled particle in bright field and fluorescent modes. Profiles of fluorescence are presented in FIG. 4B. The center of the formation particle is a bright one. This fluorescence distribution is constant for the same experimental conditions, e.g. 50 mM Mg²⁺ ion concentration.

Within the classical molecular dynamics (MD) approach, the process of M-CA assembly was analyzed in an aqueous solution (FIG. 4C) in the presence of Mg²⁺ ions

The formed M-CA-ssDNA particles have a repeated donut-like shape, average size is 35-50 μm (FIG. 4D). The particles consist of platy flakes with estimated thickness about 100 nm. The particles have pronounced growth center and several growth planes in contrast to pure melamine cyanurate crystals with needle-like shape, that suggests impact of the ssDNA on the nucleation process.

### Example 3

### Ion Regulated Particle Formation

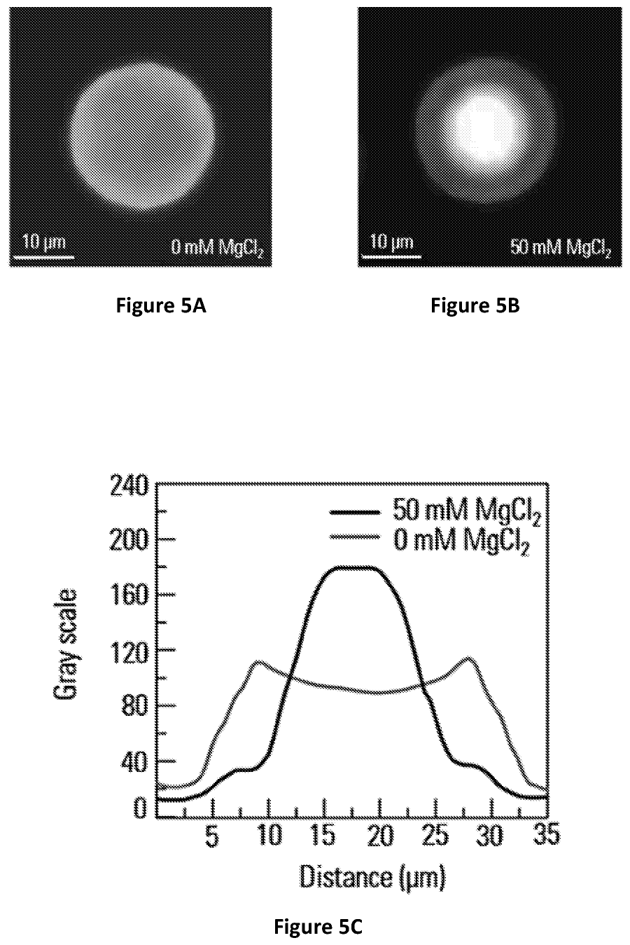

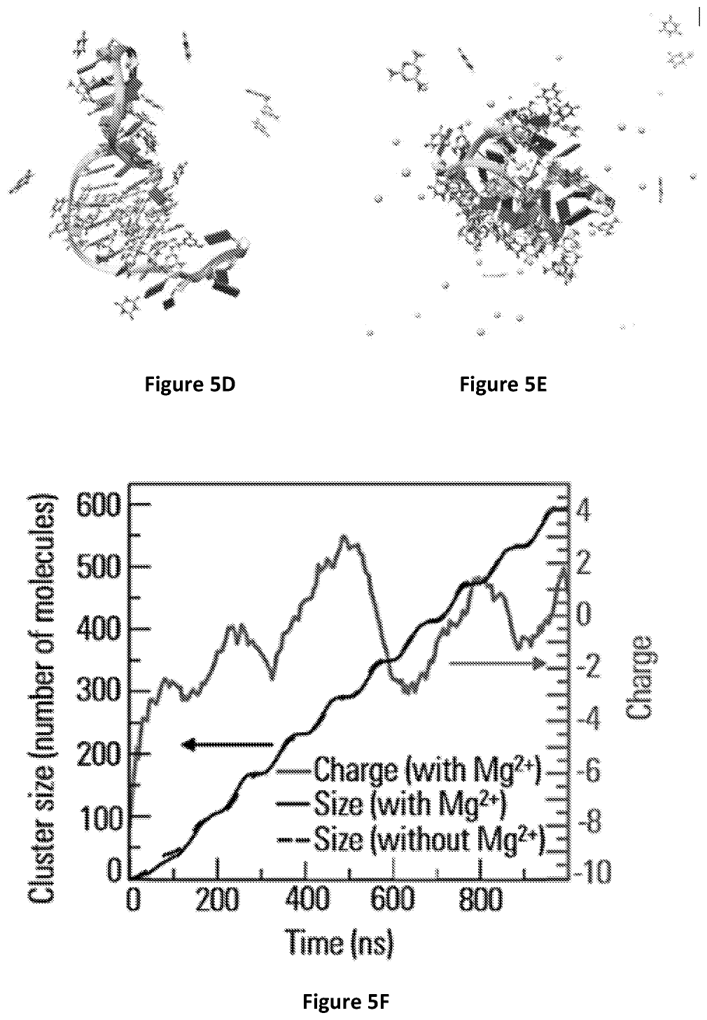

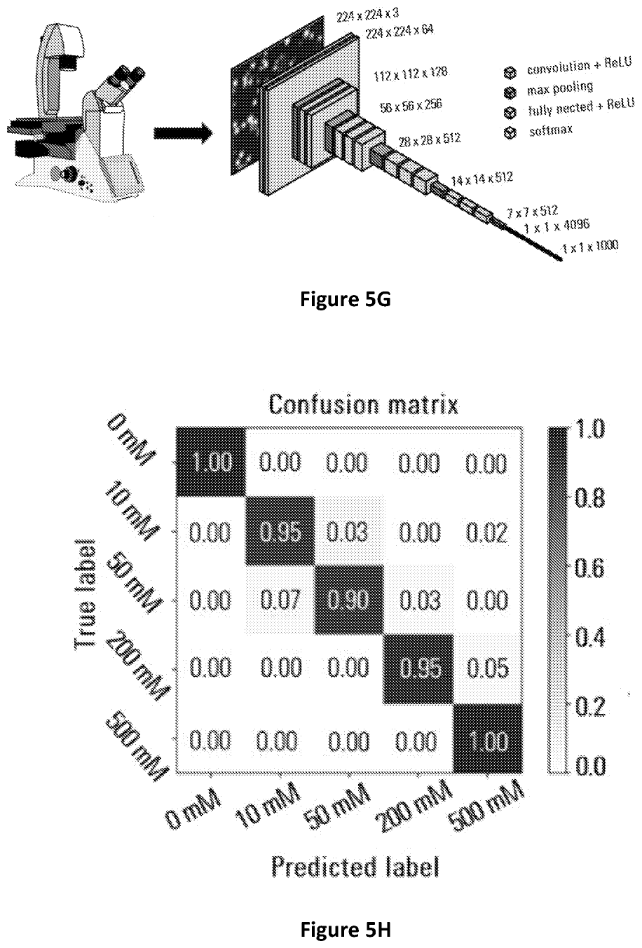

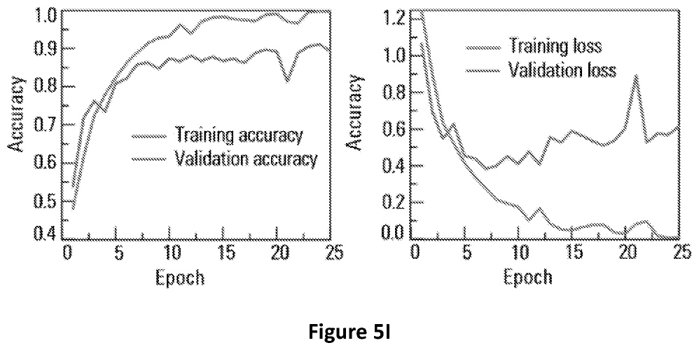

The luminescence intensity of encapsulated ssDNA and its distribution among M-CA depended on Mg²⁺ concentration (The alkaline and alkaline earth metal ions, most importantly Mg²⁺ neutralize ssDNA's negative charge). In the absence of Mg²⁺, a fluorescence intensity of M-CA-ssDNA particles was similar throughout the particle (FIG. 5A). In contrast, in the presence of 10 mM Mg²⁺, particles have pronounced bright center and pale edges in RHOD channel (FIG. 5B).

The cross-sectional profile of fluorescence intensity along the particle diameter corresponded to the DNA distribution inside the melamine cyanurate particles (FIG. 5C). The presence of Mg²⁺ during assembly of M, CA with ssDNA leads to ssDNA localization in the particle core. However, the outer layers of the particles also show emission in RHOD channel but less intensive than particles formed without Mg²⁺.

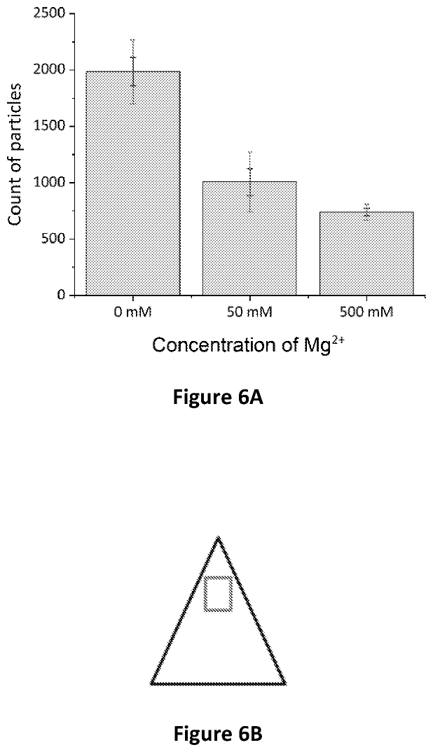

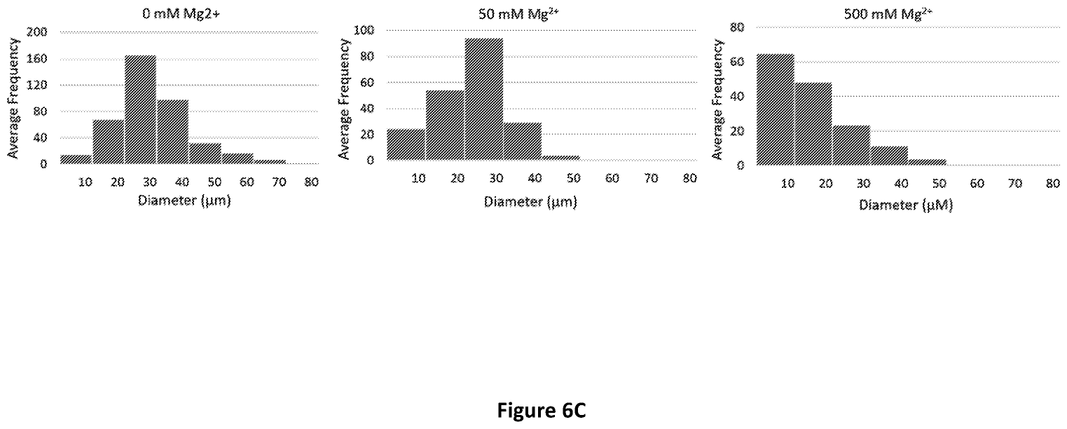

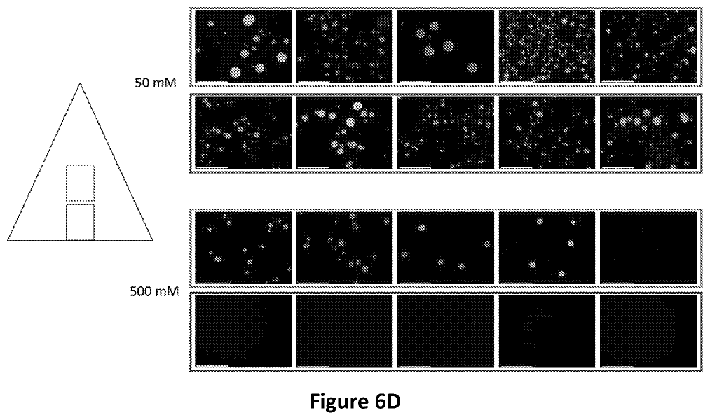

Interestingly, the concentration of Mg²⁺ greatly affected the number of forming particles. The number of particles decreased with the increase in Mg²⁺ concentration (FIG. 6A). The concentration of Mg²⁺ was varied to show the ion regulation of particle formation.

In presence of 500 mM Mg²⁺ there are no particles in the bottom section of the reaction-diffusion system after three hours of formation in contrast to the system with 50 mM Mg²⁺ (FIG. 6D). Moreover, Mg²⁺ affects the average size distribution of the particles (FIG. 6B). Particles formed in the absence of Mg²⁺ or in the presence of 50 mM Mg²⁺ had size of 20-30 μm. In contrast, the average size of the particles formed in the presence of 500 mM of Mg²⁺ was equal or less than 10 μm.

To verify Mg²⁺ incorporation in the structure of M-CA together with DNA, the local elemental analysis was used. Energy dispersive X-ray (EDX) analysis was performed for both initial particle, and one after its cutting in half ssDNA-loaded melamine cyanurate particles as Mg²⁺ can incorporate in the center of the particle through the nucleation stage when it stabilized DNA. However, no traces of Mg²⁺ were observed inside the particles.

Despite the similarities between the two systems mentioned above, high Mg²⁺ concentrations brought one significant difference to the process: they neutralized negative DNA charges during the early stages of molecular cluster formation. The total charge of the molecular cluster is close to zero in the Mg²⁺-containing system: the initial negative charge of ssDNA is compensated by Mg²⁺ ions located near its surface.

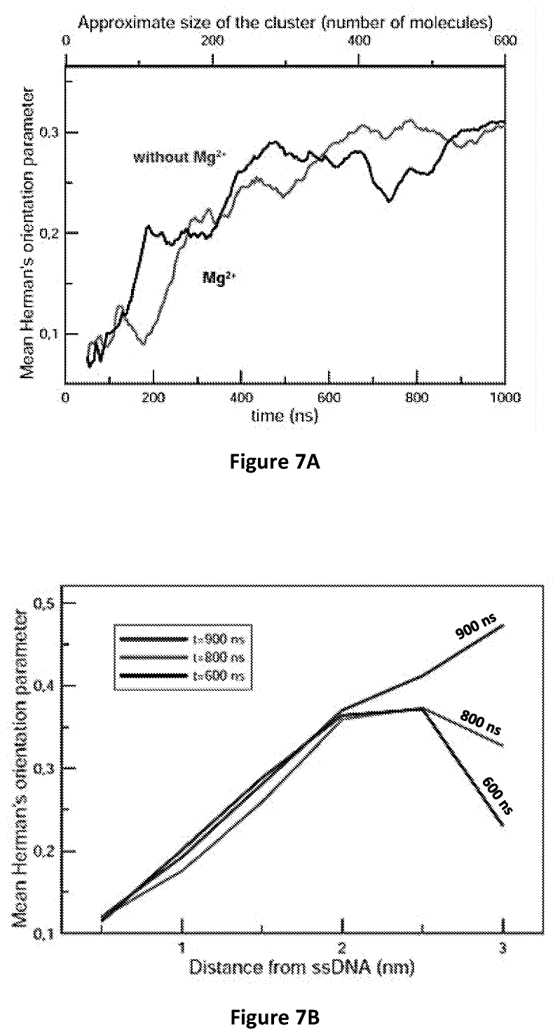

To clarify the structure of the early M-CA/ssDNA aggregates on the molecular level and to investigate the role of Mg²⁺ in the nucleation process, two microsecond-long MD simulations of M-CA-ssDNA assembly in aqueous solution were performed: one in the presence of Mg²⁺ and one without the ions. In both scenarios DNA behaves as a center of nucleation for a molecular cluster (FIGS. 5D-5E) and both growth curves are almost identical (FIG. 5F). Small oscillations of the growth curves were the result of the periodic increase in M and CA concentrations in the simulation. Analysis of the Hermans' orientation parameter (FIG. 7A-7B) demonstrated that first layers of accommodated M-CA were disordered for both systems, but after the cluster reached the size of 300-400 molecules, they became more and more organized with a pattern similar to the rosette-like (typical for pure M-CA crystals).

Hermans' orientation parameter calculation. To characterize the level of crystallinity in M-CA phase around ssDNA the Hermans' orientation factor f was calculated for every pair of molecules i and j within a cluster:

$$f = \frac{3 〈 {cos}^{2} {θ}_{ij} 〉 - 1}{2}$$

where θᵢⱼ is an angle between vectorsorthogonal to aromatic rings of the considered M or CA molecules. The f values of 1, 0, and −½ correspond to the cases of collinear, random, and orthogonal orientation.

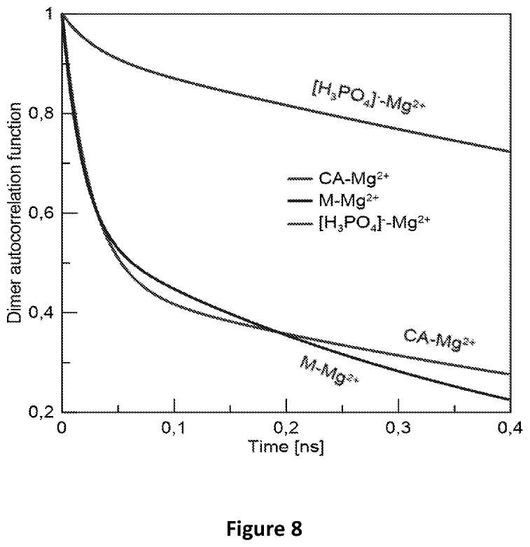

To prove that Mg²⁺ ions do not intercalate into the M-CA lattice and interact predominantly with ssDNA, additional simulation of M-CA nanoparticle nucleation was performed in the aqueous solution in the absence of ssDNA. The radial distribution function obtained between the center of mass of the formed M-CA nucleolus and Mg²⁺ ions (not shown) demonstrated that Mg²⁺ remained in the solution, i.e. M-CA nuclei were electrically neutral and did not incorporate any Mg²⁺ ions, which is in full agreement with the results of EDX analysis. For a better understanding of DNA-Mg²⁺ and M-CA-Mg²⁺ interactions in the solution, the dimer existence autocorrelation function (DACF) was calculated for the three types of molecular complexes: M-Mg²⁺, CA-Mg²⁺ and ssDNA⁻-Mg²⁺ (the last is a negatively charged phosphate group of ssDNA). Mg²⁺ did not form any long-living complexes with individual M or CA molecules, while its interaction with ssDNA is much more stable (FIG. 8).

The MD simulations demonstrated that Mg²⁺ ions did not interfere with M-ssDNA or CA-ssDNA interactions. It could imply that they affect the nucleation and formation of the particles enhancing only DNA-DNA interactions.

### Example 4

Prediction of Mg²⁺ Concentration from Fluorescent Images by Artificial Intelligence

### Deep Learning Algorithms

### Dataset

Our dataset consists of 7000 images distributed over MgCl₂ concentrations. For the neural network, 5 classes were provided: 0 mM, 10 mM, 50 mM, 200 mM, 500 mM, corresponding to the Mg²⁺ concentrations used to create the dataset, the images were placed in folders, observing their Mg²⁺ concentrations, while a part of the triangle in which the image was taken was not taken into account by the neural network. The dataset also included about 300 test images.

### Data Processing

Data preprocessing provides standardization of unbalanced data. With proper preprocessing, it is possible to increase script rate, neural network learning rate, and increase the accuracy (Olisah, C. C. & Smith, L. Understanding unconventional preprocessors in deep convolutional neural networks for face identification. SN Appl. Sci. 1, 1-12, 2019). Data preprocessing included resizing images to ImageNet standards 224×224. During preprocessing, computer vision library OpenCV-python was used.

### Data Augmentation

Data augmentation has been used in machine learning for a long time to artificially expand the dataset size. Also, after each epoch new data can enter the model, but still based on the original and only one dataset (Zhang, Y. et al. Data augmentation and transfer learning strategies for reaction prediction in low chemical data regimes. Org. Chem. Front., 8, 1415-1423, 2021). This method was used to avoid overfitting. At each new epoch, rotated or mirrored images entered the model, that provided training of neural network without any additional data. Random blur and zoom have also been added. Such data manipulations allowed training a well-working model on a relatively small dataset.

### Software and Hardware Environment

To train the model, a computer with a Geforce RTX 2060 video card, an Intel® Core™ 15-10600 CPU @ 3.30 GHz processor, 32 GB of RAM was used. To write the code, Python programming language and the Conda package manager were used. Keras and TensorFlow were used as the main framework. Interaction with the operating system was done with OS module. Auxiliary libraries were also used. NumPy was the main library for working with arrays and math operations. Libraries such as Matplotlib, Seaborn, SciPy, Scikit-learn were used for data visualization and processing.

The M-CA particles formation can be monitored by the experimental system, but the particles differ in each gel section. Systems containing Mg²⁺ ions and systems without it were visually different. However, the changes in Mg²⁺ concentration affected the reaction-diffusion system less pronouncedly. For quantitative recognition of Mg²⁺ effect convolutional neural networks (CNN) was used, originally developed to work with pattern recognition (M. Hirohara, Y. Saito, Y. Koda, K. Sato, Y. Sakakibara, BMC Bioinformatics, 19, 526, 2018). To get high prediction accuracy the transfer learning method (A. A. K. Farizhandi, O. Betancourt, M. Mamivand, Sci. Rep., 12, 4552, 2022) was also used.

For the dataset, 7,000 fluorescent microscopy images were obtained to confirm whether dependences between Mg²⁺ concentration and the structures of assembled particles and their fluorescence intensities existed. The reaction-diffusion systems contained 0 mM, 10 mM, 50 mM, 200 mM or 500 mM Mg²⁺. The concentration of Mg²⁺ affected the fluorescence distribution of the particle and particles' size and the number of particles in different sections of reaction-diffusion system. Three pretrained CNNs were used within transfer learning method to study this effect as more effective vs. traditional analytical methods.

Eighty percent of the dataset was used for training and the rest for validation. The graphic card GTX2060 Super was used to accelerate the training. In this case, VGG16 performs the best results so it was used in the architecture of this network (FIG. 5G). When testing predictive analytics, 60 images of each class are used. These images are not included in the training dataset and randomly selected. Table 1 presents data on the accuracy of each of the pretrained neural networks.

TABLE 1
Summary table for accuracy, validation accuracy, and predictive
accuracy of several pretrained neural networks used.

| | Accuracy | Validation accuracy | Prediction accuracy |
|---|---|---|---|
| VGG16 | 99.81 | 91.14 | 95.33 |
| VGG19 | 99.10 | 86.28 | 86.00 |
| ResNet50 | 89.20 | 83.75 | 85.00 |

The predictive analytics accuracy was 96% for VGG16 (FIG. 5H). Confusion matrix reflexes the relationship between true label and predictive label of each class. Thus, test accuracy for each class is observed without the respect to the number of images for class, as the results are normalized to 1. Confusion matrix demonstrates mistakes in prediction to identify which classes can be invalidly differentiated. 4% of mistaken class prediction relates to the classes with medium magnesium ions concentrations. Samples with the absence of magnesium ions and its maximum concentration are predicted without mistakes. FIG. 5I presents the correlation between the loss function and training epochs for VGG16 neural network. The error represents the mean absolute error between the values predicted by the CNN and the label values in the validation set. With the increase in the number of epochs, retraining begins, the training accuracy and validation accuracy decrease. FIG. 5I also shows that the loss function of the training set converged faster than the validation set for several of the initial training epochs. After fifteen epochs, the convergence rate of the training and validation sets decreases. The values of error and accuracy of training and validation sets are very close. This indicates that the CNN model has appropriate generalization ability and that there is no evident over-fitting phenomenon in the training.

Therefore, VGG16 is the most reliable for the encapsulation system in contrast to other CNNs.

### Example 5

### Particles' Transformation and Cascade of DNA Transformation

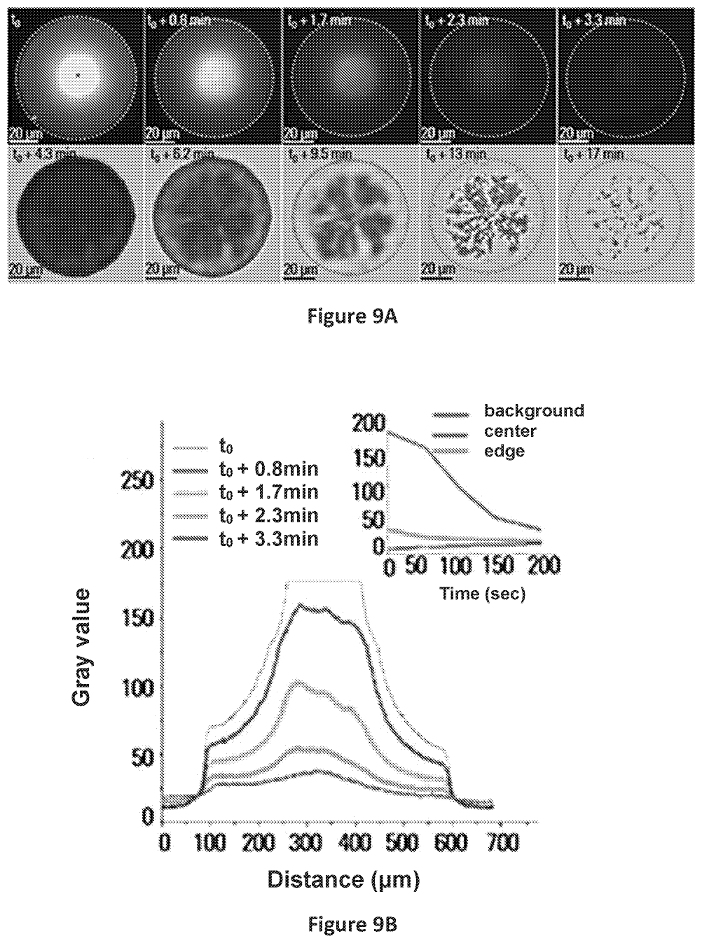

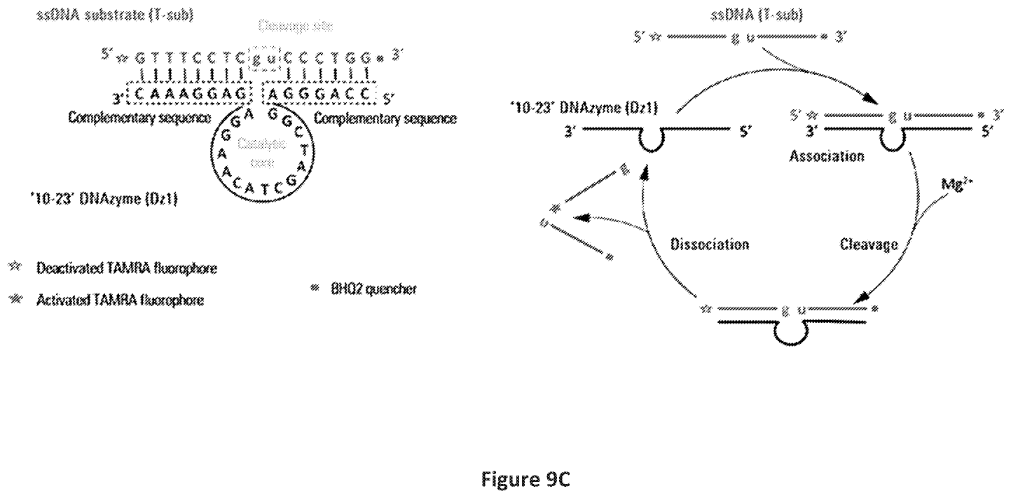

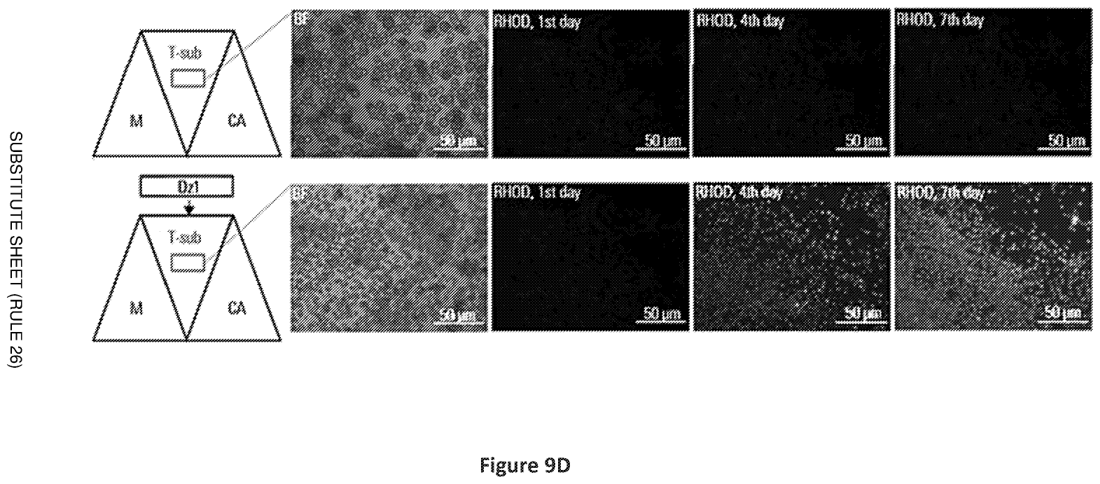

Stability of the particles relative to the environment changes was examined to define whether supramolecular particle of melamine cyanurate could be destroyed by minor perturbations. Since the M-CA supramolecular assembly is pH-sensitive, alkaline solution (NaOH, 0.5M) was added to destroy previously formed ssDNA-loaded particles (FIG. 9A). Fluorescence microscopy images of the dismantling process (FIG. 9A, 9B) show a decrease of the fluorescence in RHOD channel both in the center and on the edge of the particles. Fluorescence-intensity line scans (FIG. 9B) associated with the images indicate that the progressive loss of fluorescence intensity correlates with an increase in the fluorescence intensity of the background around the particles. Thus, the performed experiment confirms that ssDNA is released into the gel from the particles as no other reaction component besides ssDNA modified with TAMRA fluorophore is active in RHOD channel in a dissolved form. Moreover, FIG. 9A showed that DNA starts to diffuse into the gel earlier than noticeable particle destruction begins. It also indicated the post-formation ssDNA loading in M-CA particles.

It was also confirmed that ssDNA did not undergo degradation or passivation by encapsulation. To characterize functional stability of encapsulated DNA, fluorogenic reaction between ssDNA (T-sub, SEQ ID NO: 1) and DNAzyme (Dz1, SEQ ID NO: 2) was used. Dz1 consists of two DNA fragments complementary to T-sub that flank the catalytic core of 10-23 DNAzyme, responsible for the cleavage of phosphodiester bond between ribonucleotides g and u (SEQ ID NO: 1), as shown in FIG. 9C. T-sub has a fluorophore (TAMRA) and its quencher (BHQ2) attached to DNA's terminus (SEQ ID NO: 1); when the DNA strand is cleaved at g-u site, the fluorophore separates from the quencher and can generate strong fluorescence.

T-sub is entrapped in M-CA particles in the presence of Mg²⁺, and no luminescence in RHOD channel appears, even in seven days (FIG. 9D, bottom). After the formation of the particles, the gel containing Dz1 was added to the reaction-diffusion system and monitored it for 7 days. On the fourth day, luminescence in RHOD channel appeared and reached the maximum on the seventh day (FIG. 9D, top) due to T-sub cleavage and quencher BHQ2 detachment. By using hydrated metal ions to activate nucleophiles and stabilize transition states and provide site-specific attack of a 2′-hydroxyl group on the adjacent 3′-phosphorus resulting in the formation of a 2′,3′-cyclic phosphate and a 5′-hydroxyl group in a phosphate backbone of the nucleotide chain, the reaction confirms ssDNA presence on melamine cyanurate layers, its accessibility for reagents, and further applicability of M-CA in biochemical reactions.

### Example 6

### Encapsulation of RNA in Melamine Cyanurate Supramolecular Assembly

The encapsulation of the following RNA molecule labeled with carboxyfluoresceine at the 5′: 6-FAM 6-FAM-cagucgacucgccgauuagacgaaca (SEQ ID NO: 10)

Preparation of agar gels. 1 wt % agar gels containing 13 mM melamine (M), 100 nM ssDNA and 13 mM cyanurate (CA) were prepared in the presence of TBE buffer and MgCl₂ 50 mM, at room temperature.

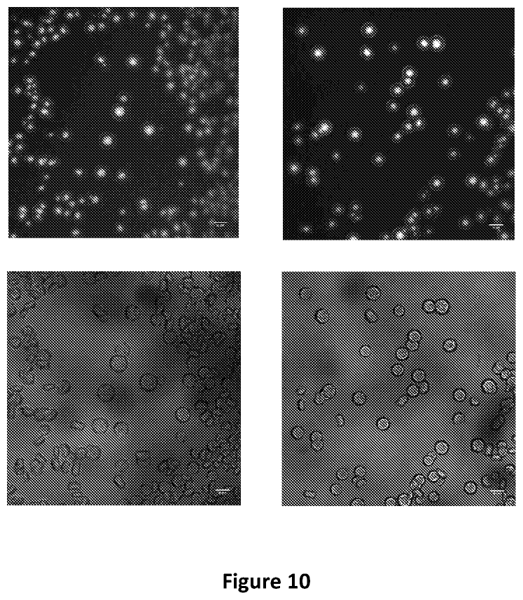

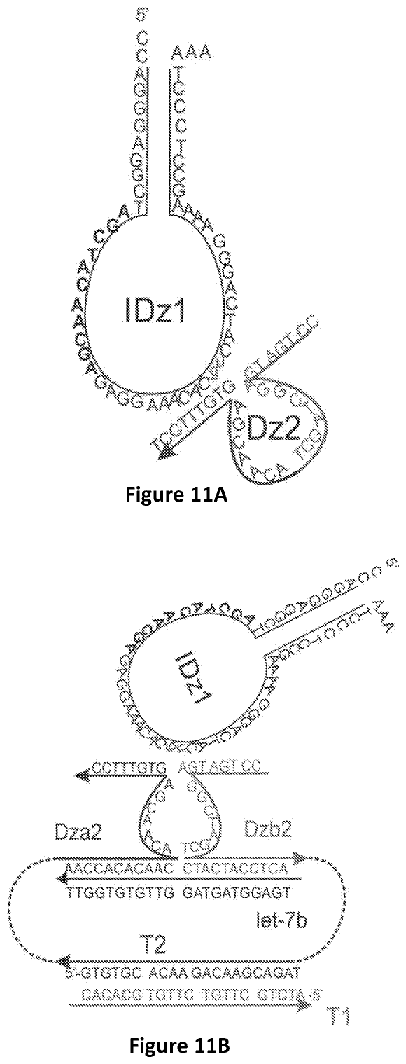

As can be seen from fluorescent (FIG. 10, top) and bright field (FIG. 10, bottom) images, RNA mostly accumulates in the center of the melamine cyanurate particle.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A supramolecular assembly comprising a solid porous particle comprising a plurality of single-stranded polynucleotides encapsulated by non-covalent organic structure.

2. The supramolecular assembly of claim 1, comprising a porous particle, wherein the porous particle is crystalline.

3. The supramolecular assembly of claim 1, wherein the non-covalent organic structure comprises melamine-cyanurate or melamine-barbiturate or derivative thereof.

4. The supramolecular assembly of claim 1, wherein the non-covalent organic structure interacts with the single-stranded polynucleotides by hydrogen bonds.

5. The supramolecular assembly of claim 3, wherein the single-stranded polynucleotides are encapsulated by melamine-cyanurate or derivative thereof.

6. The supramolecular assembly of claim 3, wherein the single-stranded polynucleotides are encapsulated by melamine-barbiturate or derivative thereof.

7. The supramolecular assembly of claim 1, wherein the assembly comprises a plurality of single-stranded DNA (ssDNA) or single-stranded RNA (ssRNA) polynucleotides, or combinations thereof.

8. (canceled)

9. The supramolecular assembly of claim 3, wherein the molar ratio between the melamine and the cyanurate is 1:1; or the molar ratio between the melamine and the barbiturate is 1:1.

10. (canceled)

11. The supramolecular assembly of claim 1, wherein the single-stranded polynucleotides comprise one or more different single-stranded polynucleotides.

12. The supramolecular assembly of claim 1, further comprising an alkaline or alkaline earth metal ions to neutralize the assembly.

13. (canceled)

14. The supramolecular assembly of claim 1 wherein the single-stranded polynucleotides comprise a fluorophore or a quencher or a spacer.

15.-18. (canceled)

19. The supramolecular assembly of claim 1, wherein the single-stranded polynucleotides are released upon applying a pH of between 3-10 and/or a temperature of between 40-80° C.

20. A composition comprising the supramolecular assembly of claim 1.

21. (canceled)

22. The composition of claim 20, wherein the composition is a vaccine, diagnostic composition, or analytical composition.

23. A method of detecting the presence of a nucleotide sequence in a sample, the method comprising:
a. obtaining a sample;
b. contacting the sample with a supramolecular assembly comprising a solid porous particle comprising a plurality of single-stranded polynucleotides encapsulated by non-covalent organic structure;
c. adjusting the pH of the solution for dissolution of particles; and
d. detecting the presence of a nucleotide sequence.

24. (canceled)

25. The method of claim 23, wherein sample is a liquid sample.

26. The method of claim 23, wherein the sample is obtained from a bodily fluid or a water source.

27. (canceled)

28. The method of claim 23, wherein the nucleotide sequence is derived from one or more of an animal, a human, a plant, a cultured cell, a microorganism, a virus, a bacterium, or a pathogen.

29. The method of claim 28, wherein the pathogen is a bacterial, fungal, or viral pathogen.

30. The method of claim 23, wherein the nucleotide sequence is selected from one or more of DNA, RNA, cDNA, mRNA, CRNA, tRNA, ribosomal RNA, dsDNA, ssDNA, miRNA, siRNA, and mitochondrial DNA.

31. The method of claim 23, wherein adjusting the pH of the solution for dissolution of particles comprises applying a pH of between 3-10 and/or a temperature of between 40-80° C.

32. The method of claim 23, further comprising the step of concentrating the contacted solution.

33. A method of gene suppression in a cell, the method comprising: contacting the cell with a supramolecular assembly comprising a solid porous particle comprising a plurality of single-stranded polynucleotides encapsulated by non-covalent organic structure.

34. A method of expressing a gene in a cell, the method comprising: contacting the cell with a supramolecular assembly comprising a solid porous particle comprising a plurality of single-stranded polynucleotides encapsulated by non-covalent organic structure.

35. The method of claim 33, wherein the cell is a plant cell.
